(19) United States
(12) Patent Application Publication
(10) Pub. No.: US 2025/0277181 A1
(43) Pub. Date: Sep. 4, 2025

(54) SYSTEMS AND METHODS FOR INTERFACING WITH LIVING IN VITRO BIOLOGICAL MATERIAL

(71) Applicant: The Board of Trustees of the University of Illinois, Urbana, IL (US)

(72) Inventors: Mattia GAZZOLA, Urbana, IL (US); Xiaotian ZHANG, Champaign, IL (US); Zhi DOU, Urbana, IL (US)

(73) Assignee: The Board of Trustees of the University of Illinois, Urbana, IL (US)

(21) Appl. No.: 19/069,882

(22) Filed: Mar. 4, 2025

Related U.S. Application Data
(60) Provisional application No. 63/561,077, filed on Mar. 4, 2024.

(51) Int. Cl. C12M 1/34; C12M 1/36; C12M 3/00; C12N 5/0793

(52) U.S. Cl. CPC C12M 41/46; C12M 21/08; C12M 41/48; C12N 5/0619

(57) ABSTRACT

Provided are systems and related methods for at least electrically interfacing with a biological material. The systems may be fully automated via use of a robotic positioner configured to facilitate physical handling, accessing and interfacing any one or more of a plurality of independent biological materials. A multi-electrode array algins and electrically connects with a printed circuit board that is positioned by the robotic positioner.

### CROSS-REFERENCE TO RELATED APPLICATIONS

This application claims the benefit of priority to U.S. Provisional Patent Application No. 63/561,077, filed Mar. 4, 2024, which is hereby incorporated by reference to the extent not inconsistent herewith.

### STATEMENT REGARDING FEDERALLY SPONSORED RESEARCH OR DEVELOPMENT

This invention was made with government support under Award Numbers 2123781 and 1830881 awarded by the National Science Foundation. The government has certain rights in the invention.

### BACKGROUND OF INVENTION

There is a need in the art for accurate, modular, fully customizable and portable systems that can reliably electrically interface with biological tissue, including neuronal cells. This is important for a number of applications, including neuroscience research and neural computing, for example.

Conventional systems are limited in that they are difficult to customize with minimal to no customization ability. Furthermore, those systems are costly, particularly for multi-channel systems. For example, the MEA2100-Systems (www.multichannelsystems.com/products/mea2100-systems).

The above limitations are addressed herein by incorporating base components, including the MEA, PCB, and support materials that are either off-the-shelf or are readily customizable, that can be readily swapped out, depending on the application of interest. The special robotic positioner with software control provides the ability to massively multiplex and increase throughput compared to the costly and non-configurable conventional systems.

### SUMMARY OF THE INVENTION

Provided herein are versatile, scalable and reconfigurable systems for electrically interfacing with biological tissue, including cultured cells. The systems are applicable over a range of applications, particularly as the systems are compatible with any biological material where it is desirable to have an electrical interface. The systems provided herein are constructed from cost-effective parts and using standard processing techniques. For example, the microelectrode array (MEA) is readily configured to have any number of electrodes, including up to 512 or more electrodes. The systems provided herein can have custom and interchangeable printed circuit boards, selected to interface with the MEA, with the number of channels matching the number of electrodes of the MEA. The systems can integrate with other interfaces beyond electrical, including optical, fluidic and alignment interfaces. Genetic optical markers can be combined with the high-temporal and high-spatial resolutions provided by the “base” platform configuration.

Full automation is obtained by incorporation of a robotic positioner (e.g., a robotic arm) for handling of multiple platforms in a single cluster. Software-based interface is used to provide handling of robotic positioner with full and complete interfacing over any of the plurality of platforms. This is particularly advantageous with respect to increased efficiency and throughput compared to conventional systems requiring more user intervention.

The built-in modularity by the configuration of core components facilitates the efficient reconfigurability and seamless integration with standard stimulation, imaging and processing tools.

Central to the systems is a reconfigurable platform that hosts MEA-chips and custom printed circuit board (PCB) interfaces to form a complete signal chain, from neural substrates to data acquisition terminal and downstream processing. Designed with built-in modularity, the platform supports a variety of commercial/custom MEAs entailing up to 512 or more channels, while allowing seamless integration with electrical and optical stimulation as well as fluidic interaction. The reconfigurability also allows the systems to work across multiple industry standards. A custom MEA fabrication process can leverage maskless photolithography, favoring the rapid prototyping of MEAs across configurations, spatial topologies, and constitutive materials.

Through a dedicated analysis and management software, the system utility is demonstrated across multiple neural cultures, including embryonic stem cell-derived and primary neurons, organotypic brain slices and 3D engineered tissue mimics. To showcase the robustness and versatility of the systems provided herein, a series of characterizations and applications are illustrated including multimodal stimulation, fluidic manipulation, concurrent calcium imaging and long-term recording.

Overall, the system can be used as a customizable in vitro neuron recording, stimulation and analysis solution, that can be widely deployed, including due to its affordable (>10X cost reduction against commercial products).

The systems described herein are compatible with a single “recess feature”, including in a less than fully-automated manner. For example, a sterile platform having a single recess feature with a single removable lid. The MEA may support growth of one biological material where the electrical interfacing is confined to that biological material within the recess feature. While the components generally remain the same as for the array of recess features, there is no need for a robotic positioner that moves over a plane of the sterile platform. For example, the lid can be removed by hand and the PCT interface manually contacted with the MEA. As but one example, the lid can be hinged in a manner that the lid can flip open and closed by a user. The PCB interface may be incorporated into the lid. In this manner, long-term electrical interfacing with a biological material may be readily and reliably accomplished without a need for a robotic positioner.

The appended claims are specifically incorporated by reference herein.

Provided is an automated system for electrophysiologically interfacing with a biological material. For example, the systems may have a sterile platform comprising a plurality of recess features, each recess feature configured to support a biological material. A microelectrode array (MEA) can be positioned in each of the plurality of recess features and is configured to electrically interface with a biological material. Electrical interconnects, including interconnects supported by the MEA, are configured to electrically interface with the biological material. A downstream signal acquisition unit is electronically connected to the biological material via the electrically conductive interconnects, including contact pads connected to microelectrodes by interconnects. A plurality of lids can cover each recess feature. That is, each recess feature can have a distinct lid removably connected to removably seal the biological material from a surrounding environment. The high number of unique electrical interface channels can result in a relatively high amount of data being transmitted to the biological material (e.g., electrical stimulation) and/or being generated by the interfacing (e.g., recording of electrical parameters). Accordingly, provided is a processor configured to receive instructions from a user for electrically interfacing with the biological material. A robotic positioner has an end effector with a printed circuit board (PCB) interface to electrically connect to the MEA and a lid grip to reversibly engage with each of the plurality of lids. An optical detector can be used to align the PCB and the MEA and ensure good and reliable electrical interfacing. A controller is utilized to implement an electrophysiological interface scheme between the robotic positioner interface unit and at least one of the MEAs positioned in the recess feature. The electrophysiological interface scheme may include transmitting a position signal to the robotic positioner to position the end effector and deploy the lid grip to engage the lid of a selected recess feature and transmitting a removal signal to the end effector to remove the lid from the recess feature. An interface signal may be transmitted to the robotic positioner to align the PCB interface with the MEA and electrically connect the PCB interface with the MEA, thereby electrophysiologically interfacing with the biological material.

An optical light source can be connected to the end effector and configured to illuminate the MEA for the optical detector and alignment of the PCB interface with the underlying MEA.

The biological material can be positioned within a container and the container positioned within the recess feature, wherein each container is removable and replaceable. In this manner, quick, efficient and automated biological material can be facilitated.

The plurality of recess features can be provided in an array having 12 or more of the recess features configured for independent electrophysiological interfacing with the biological materials in each of the recess features.

The MEA may comprise up to 512 electrode channels, with each electrode channel electronically connected to the PCB.

The MEA may comprise a spatial array of electrodes patterned on a biocompatible surface for directly supporting growth of a biological cell of the biological material. The MEA may be a customized chip configured to match with the PCB that is a swappable PCB configured to an application of interest.

The electrical interconnects may be provided on the MEA and physically connect each microelectrode that is positioned in a central region, wherein the biological material is positioned to a corresponding plurality of contact pads positioned in an outer region perimeter orientation where the PCB interface makes electrical contact.

The controller may comprise a computing device, the computing device including: a processor, and memory communicatively coupled to the processor, and the computing device implements the electrophysiological interface scheme for the automated system. The memory may include a non-transitory computer readable medium storing processor-executable instructions encoded as software, which, when executed by the processor, cause the processor to implement the control scheme for the system. The automated system is compatible with a range of electrophysiological interface schemes, including to record electrical output from the biological material via the MEA; provide an electrical activation input to the biological material via the MEA; and/or to record electrical output from the biological material via the MEA and provide an electrical activation input to the biological material via the MEA.

The electrophysiological interface scheme may further include: removing the biological material from the recess feature; and inserting a different biological material into the recess feature; so that the automated system comprises a fully reconfigurable platform.

The electrophysiological interface scheme may further include controlling one or more cell culture parameters to support or maintain growth of the biological materials in the recess features, such as temperature (T), humidity, CO₂ concentration. This can be implemented with an incubator electronically connected to the system.

The automated system is compatible with a range of biological materials. Examples include, but are not limited to neuronal cells, cardiac cells, stem cells, brain cells, tissue slices, skeletal muscle cells, retinal ganglion cells, multi-cell type co-cultures including neuromuscular junctions and the combination of multiple neuronal subtypes, a bioengineered tissue, and ex vivo slices of a biological tissue.

The automated system can have an optical interface, a fluidic interface, or both an optical and a fluidic interface, including automated interfaces controlled by computer-implemented software. For example, an optical source can be operably connected to the controller for implementing the optical interface, including for detecting an optical marker and/or activation of an optical probe. The fluidic interface may comprise an inlet tube fluidically connected to the plurality of recess features for introducing cell media from a source of cell media to the biological material in the recess feature.

The automated system is compatible with any of a range of recess feature number, size, and geometry. For example, the recess features may be cylindrical having a depth and a diameter, wherein the depth is between 0.5 cm and 3 cm and the diameter is between 1 cm and 15 cm.

Also provided herein are methods of electrophysiologically interfacing with a biological tissue using any of the automated systems described herein. The method may comprise the steps of: inserting a biological material onto a microelectrode array (MEA) positioned within a sterile platform recess feature; covering the inserted biological materials in the sterile platform recess feature with a lid; positioning the sterile platform into an incubation chamber; controlling the incubation chamber to maintain viability of the biological material for a culture time period and to thereby electrically connect the biological material with at least one microelectrode of the microelectrode array; providing an electrophysiological interface scheme to control a robotic positioner having an end effector with a lid grip and a PCB interface for removing at least one lid with the end effector lid grip from at least one underlying recess feature to generate a lid in a lid removed configuration and thereby provide an opening to access the biological material; aligning the PCB interface with the MEA (including by an optical detector controlling the positioned of the end effector); interfacing the robotic positioner PCB interface with the biological material by contacting the PCB interface with the MEA for an interface time period; terminating the interfacing by removing the PCB interface unit from the MEA; directing the robotic positioner lid grip to contact the lid in the lid removed configuration and move the lid over the underlying recess feature; depositing the lid to cover the underlying recess feature and thereby seal the biological material from the surrounding environment and generate the lid in a lid sealed configuration. The steps may be repeated any number of times for any number of additional recess features.

The method may further comprise the steps of controlling a cell culture parameter to maintain viability of the biological material, wherein the cell culture parameter is selected from the group consisting of: a media flow rate; a temperature; a humidity; a CO₂ concentration; and any combination thereof. This control is automated in that it can be software implemented to provide the necessary commands via sensors and feedback loops to ensure the cell culture parameter remains within a biologically-suitable range.

The method may further comprise the step of directing via the electrophysiological interface scheme the robotic positioner to replace at least one biological material within a recess feature with a replacement biological material.

Also provided is a system for electrophysiologically interfacing with a biological material comprising: a sterile platform; a microelectrode array (MEA) supported by the sterile platform, the MEA comprising: a plurality of electrodes positioned in a central region of the MEA configured to support and electrically connect with the biological material; a plurality of contact pads positioned around a perimeter region of the MEA; a plurality of electrical interconnects, wherein each of the plurality of electrodes is connected to a unique contact pad by one of the electrical interconnects; a PCB interface, wherein the PCB interface is configured to reversibly connect to the plurality of contact pads and configured to provide electrical connection between the PCB interface and the biological material; and a downstream signal acquisition unit electronically connected to the PCB interface.

The system may further comprise: a cover to cover the MEA supporting the biological material; a downstream signal acquisition unit; a cable that electronically connects the PCB interface to the downstream signal acquisition unit; wherein: the PCB interface is connected to the end effector to provide a PCB interfaced with the biological material and a PCB not-interfaced with the biological material condition; the cover is configured for movement via the end effector.

The PCB interface may be a swappable PCB and the MEA may be a custom chip having a user-selected number of contact pads, with the swappable PCB selected based on the number of contact pads, including a number of contact pads ranging from between 12 and 600.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIGS. 1A-1E: System overview. FIG. 1A is a computer-aided design (CAD) illustration of our recording platform, assembled from low-cost materials and commercially available parts. The platform is designed to be compatible with a series of PCB options across a variety of different recording capacities. The full list of components used to construct the hardware system can be found in Table 1. FIG. 1B is an assembled 128-channel recording platform. FIG. 1C is a stimulation setup. Electrical stimulation can be performed by connecting a pulse generator (e.g., Stimjim) to extended pogo pins through a direct connection. Alternatively, for finer control, the Open Ephys terminal can be replaced with an Intan terminal, enabling bidirectional recording/stimulation across up to 128 channels. Optical stimulation is achieved by placing optical fibers on either side of the MEA. FIG. 1D is a lab deployment of the recording system. Recordings are performed inside an incubator, while the data acquisition board and various external modules are set outside of the incubation environment. FIG. 1E contains an overview of the customized software package for data analysis, including an interface module for loading continuous or binary data recorded using the Open Ephys GUI, along with a variety of commonly utilized functions relevant for data processing and network characterization.

FIG. 2: Comparison among recent custom electrophysiology approaches. [29-32].

FIGS. 3A-3G: MEA-chips fabrication, design, and characterization. FIG. 3A is an overview of the MEA fabrication process using cleanroom techniques. FIG. 3B contains custom MEA designs of variable capacity (59, 128, 256, and 512 electrodes). Electrodes are 30 μm in diameter. The 59-electrode version replicates the design of the widely employed commercial multichannel system 60MEA200/30 series. To maximize the use of recording channels on the amplifier chip, while enabling the use of the low-cost external stimulator Stimjim, four additional electrodes are specifically dedicated to electrical stimulation (except for the 59-channel version). Located outside each edge of the standard electrode grid, stimulation at these sites allows us to probe effects of localized neural activation. FIG. 3C contains three 128-channel MEA 40 designs with customized topologies. The curved layout presents a curvature of ˜0.35 mm⁻¹, matching the typical morphology of mouse cerebral cortex. [66] The perturbed layout entails random electrode displacements of up to 50 μm from the standard, rectangular layout of FIG. 3B. FIG. 3D shows platinum black deposition reduces electrodes impedance. Microscopic images are taken after deposition for both 30 and 5 μm electrodes. Close-ups illustrate the granular structure that Pt forms on the electrode. FIG. 3E Impedance magnitude before and after Pt deposition across electrodes of different sizes. FIG. 3F Time-deposition dependent impedance magnitude reduction. FIG. 3G Time-deposition dependent change in impedance phase.

FIGS. 4A-4G: 2D neural systems. FIG. 4A Fluorescence microscopic image of eGFP ESC-MNs seeded on MEA. FIG. 4B Raster plot shows the spontaneous neural activity recorded from a rectangular MEA layout (FIG. 3B) of 30 μm-electrode on day 17 after seeding. Three seconds of filtered data from a representative channel reveal individual spikes as well as a burst event. Spikes are overlaid after sorting. FIG. 4C Averaged firing rates across all channels over 2 min, as a function of days after seeding. Results from MEA chips of 30 and 5 μm electrodes are reported. Error bars indicate standard deviation across channels. FIG. 4D Averaged firing rate of each recording channel on day 17. The dataset is used to calculate the signal-to-noise ratio (SNR), defined as the ratio between the mean spike peak amplitude and the standard deviation of the full signal. FIG. 4E Spatial activity propagation in response to localized electrical stimulation, at two different stimulation sites (top and bottom) and with two different intensities (700, 1500 mV). Each stimulation experiment entails 6 to 8 trains of 20 Hz biphasic stimulation pulses, with each train lasting for 1 s. Biphasic pulses present a positive-to-negative transition, where each phase has a duration of 400 μs. These parameters are selected based on ref. [74]. Data are recorded from the 30 μm MEA of FIG. 3b, on day 17 for the 700 mV tests and on day 19 for the 1500 mV test. FIG. 4F ESC-MNs cultured on a 4-well MEA. Spontaneous activity is recorded (left raster plot) and channel-wise correlation coefficients are computed to demonstrate separation between different clusters. We then proceed with selective optical stimulation of well #3. Stimulation lasts 1 min with a 1 s pulse train delivered every 5 s. Each train is comprised of pulses at 40 Hz, 20% duty cycle and 1 A current amplitude. The experiment was performed on day 13 using a 465 nm LED source acquired from Doric lenses. FIG. 4G Long-term recording for 24 h. Box plot shows the spontaneous activity recorded for the first 12 min of every hour. The red dash denotes the median value, the box indicates the range from the first quartile (Q1) to the third quartile (Q3), and the vertical line extends from the box by 1.5 times of the inter-quartile range (IQR=Q3-Q1). Outliers beyond the range of the vertical line are not plotted.

FIGS. 5A-5H: Higher dimensional neural systems. FIG. 5A Platform integration with an add-on fluidic interface module for brain slice recordings. The add-on module is made of bio-compatible stereolithography material using a Formlabs 3D printer. Before the experiment, the printed parts are autoclaved and sanitized to ensure nontoxicity to the brain tissue. Hypodermic needles used for fluid flow are grounded to the recording system to minimize noise levels. FIG. 5B Microscopic images of organotypic brain slices placed on curved MEAs with either 30 or 5 μm electrode diameters. Electrodes are arranged to match the curvature of the cortex region, which is the shaded area around the center of the slice. FIG. 5C Spike raster of spontaneous tissue activity, measured around 10 min after transferring onto the MEA. A raster plot corresponding to electrically stimulated activity, measured around 15 min after transferring, is also reported. Each red dashed line indicates a single biphasic pulse (applied at the bottom stimulation site). Snapshots of filtered neuronal signals during spontaneous recording showcasing single-unit and multi-unit activities. Cutouts are plotted to demonstrate the detection of characteristic spike shapes. FIG. 5D Firing heatmaps of spontaneous and stimulated activities. FIG. 5E 3D engineered neural tissue mimics (NTMs) made of cell-ECM mixture. Custom multi-well MEA hosts PDMS molds of four different shapes, demonstrated in various controlled NTMs geometries. GFP signals illustrate motor neurons distribution within each tissue. FIG. 5F Spontaneous activity recorded on day 7, from an NTM sample seeded on a 128-electrode, rectangular MEA. FIG. 5G Engineered neural spheroids. Microscopic images are taken to visualize free-floating spheroids and after transferring onto a commercial MEA (used here to confirm cross compatibility with our platform). FIG. 5H Spontaneous activity recorded from the same sample.

FIGS. 6A-6B: Alteration of the recording platform to facilitate concurrent electrophysiology and microscopic imaging. FIG. 6A Placing of the recording device inside an inverted microscopic chamber. A thin layer of aluminum mesh is fabricated to wrap around the device and serve as Faraday cage. Indium tin oxide (ITO) MEAs and PNs are utilized in this experiment. FIG. 6B Examples of simultaneously acquired fluorescence signal and electrophysiology data. Video analysis is performed within a ROI of 0.57×0.82 mm, while electrical data are recorded from a channel located near the center of the ROI. Left inset: A peak in the ΔF/F0 signal and a LFP event share similar temporal characteristics, where a rapid initiation period (<150 ms) is followed by a gradual restoring phase (1 s for LFP and 3 s for ΔF/F0). We align the two signals according to their first edge. Right inset: Snapshots of the fluorescence signal before and during a burst event.

FIG. 7: Comparison between our customized, passive electrophysiology solution with commercial HD-MEA (high-density MEA) systems. Costs are based on acquired quotations.

FIG. 8: Illustration of the MEA micro-fabrication process using standard cleanroom technologies.

FIG. 9: Image of the MEA with four different electrode sizes used for impedance characterization.

FIG. 10A show PDMS wells of various shapes for neuron seeding. FIG. 10B Using 3D-printed stoppers for centering different MEAs.

FIG. 11A Microscopic image of PNs seeded on a 128-channel MEA with perturbed layout and 30 μm electrodes. FIG. 11B Raster plot shows the spontaneous neural activity recorded 15 days after seeding. Snapshots of filtered data from representative channels. FIG. 11C Averaged firing rate and SNR of each recording channel on day 15. FIG. 11D Spatial activity propagation in response to localized electrical stimulation at two different stimulation sites (top and bottom). Stimulation is also performed on day 15, with intensity of 1500 mV.

FIG. 12A Transparent MEA with 100 nm ITO layer deposited on a BOROFLOAT 33 (BF33) glass wafer. FIG. 12B Transmittance of the ITO MEA at different wavelengths. Measurements of the plain glass wafer are also plotted for comparison.

FIG. 13A Deployment of a system in a remote lab. FIG. 13B Connecting the recording platform with a dual channel peristaltic pump for culture media circulation.

FIG. 14A Four recording platforms with increasing number of recording channels. FIG. 14B Spontaneous recordings using the 512-channel device and MEA.

FIGS. 15A-15B System replication and remote deployment. FIG. 15C System validation through the recording of the spontaneous activity of an ESC-MNs culture.

FIG. 16A is one embodiment of an automated system that is useful as a robotic neural interface. The system can have a geometric footprint configured for positioning within an incubator to support biological material, including a cell culture, including an outer chamber that is functionally constructed to work as an incubator. FIG. 16B illustrates a system's robotic positioner (including end effector) for electronic manipulation of the MEA and biological material. The robotic positioner having an end effector with a lid grip can control the lid in a lid-removed configuration or a lid sealed configuration.

FIG. 17 is a photograph of a system in an incubator with controller module (right panel) and a close-up view of the MEA chamber where the electrodes are electrically connected to biological cells, with lid holders and end effector of robotic positioner that moves along a gantry system for lid placement for lid removal and attendant ready for electrical interfacing of one recess feature.

FIG. 18 is an exemplary software architecture for robotic and environmental control.

FIG. 19 are photographs illustrating the working flow of the robotic positioning and recording of the bio-sample. The PCB of the robotic positioner end effector in contact with the contact pads of the MEA electrically interface with the biological material supported by the electrode region of the MEA. The system initializes (top left “initialization”) and calibrates (bottom left “calibration)). The chamber is opened with lid removal (top middle “open chamber”) and there is electrical interfacing between the MEA and biological material (bottom middle “record”). The robotic positioner places the lid over the recess feature (top right “place lid”) and upon completion of interfacing with each recess feature, the robotic positioner closes the chamber (bottom right “close chamber”).

FIG. 20 summarizes the optical imaging and image processing for precise end effector alignment via pattern detection and corresponding center location determination. Such a process ensures good alignment between the robotic arm end effector and underlying biological tissue interfaced with MEA.

FIG. 21 provides sample data recorded using the robotic arm end effector interface. The left graph is a plot of channel output over time. The middle plots are voltage traces for three channels. The right graph is a plot of signal-to-noise ratio for each channel.

FIG. 22 is a schematic illustration of an exemplary automated system for electrophysiologically interfacing with a biological material.

FIG. 23 is a close-up highly schematized view of the components and processer associated with the implementation of an electrophysiological scheme for lid positioning, electrical interfacing with tissue, and attendant control and data collection.

### DETAILED DESCRIPTION

In general, the terms and phrases used herein have their art-recognized meaning, which can be found by reference to standard texts, journal references and contexts known to those skilled in the art. The following definitions are provided to clarify their specific use in the context of the invention.

“Electrophysiologically interface” refers to the electrical connecting with a biological material, such that there is at least one-way and, more preferably, bi-directional communication with respect to an electrical parameter, while maintaining the ability to support ongoing growth or status of a biological material that comprises living biological cells and/or tissue. The interface provides the ability to impart an electrical parameter to the material and/or measure an electrical parameter of the material. The electrical parameter maybe, for example, an electric potential at one or more locations and/or an electric potential field over a relatively large area corresponding to the MEA footprint. Depending on the application of interest, the electrical parameter may be measured over a time course, such as ranging from the order of seconds to minutes or hours, and any subrange therein.

“Biological material” refers to an in vitro material that contains one or more living cells. Biological material is used broadly and can encompass a cell culture, a plurality of cell types that are in culture, a tissue isolated from an organism or animal, a bioengineered material that is a mixture of living cells, support scaffold and artificial material, including engineered cells and/or engineered scaffold. The biological material may be further characterized as an electroactive biological material, referring to biological material having relevant electrical properties, or that may respond to an electrical actuation signal, and so are fairly characterized as being compatible with an electrical interface as described herein.

“Sterile platform” refers to a substrate in which a biological material may be supported for a time period without contamination that would adversely impact the system. In particular, the biological material may be accessed, including repeatedly accessed over a period of time, in a manner such that contamination is avoided. The platform, therefore, at least is sterile in a region of the biological material. Other regions of the platform separated from the biological material may have relaxed sterility constraints.

“Recess feature” refers to a region of the sterile platform that is configured to contain or hold a biological material and related components useful for establishing an electrical interface. The recess feature may correspond to a well, including a cylindrical well having a depth and a diameter, a square or a rectangular well. The recess feature may be a plurality of recess features arranged in an array. The recess feature may have any shape, so long as the ability to reliably electrically interface and support the biological material is not adversely impacted.

The term “interconnect” refers to a component that connects otherwise physically separated components in a manner that does not adversely impact each component or material functionality.

The term “electrical interconnects” refers to an interconnect that electrically connects two or more components, such that a change in an electrical parameter of one component is conveyed to another component. In this manner, an electrical interface is established between the otherwise electrically isolated components. The interconnect may be provided in a track configuration embedded in a material or component, such as within a MEA. The interconnect, in this manner, can then facilitate an electrical interface between the biological material that is grown on the MEA and a downstream signal acquisition unit. This is particularly useful for a downstream signal acquisition unit that may not be compatible with being located in an environment suitable for tissue culture that has a relatively high temperature and humidity. The electrically conductive interconnects are formed of a material that is capable of conducting electricity such as, for example, a metal or metal-containing material.

“Robotic positioner” refers to a component that is controllably positioned by a user, preferably an electronic controller, to take one or more actions with respect to one or more electroactive biological materials. The robotic positioner may have an end effector. “End effector” refers to a movable portion of the robotic positioner to which one or more components are connected that are useful for automation. For example, a lid grip that can reliably and repeatedly engage and disengage with a lid to provide a lid-removed and a lid-engaged, thereby providing access to the underlying biological material by gripping and removing the lid and culture-ready biological material by reliably covering the recess feature with the lid. This can be repeated for an array of recess features having a corresponding array of lids. Similarly, an “optical detector”, including a camera or an imager, may be used to ensure a PCB interface is properly aligned with MEA in the underlying recess feature. With alignment, the robotic positioner may then contact the PCB interface with the MEA, thereby establishing electrical contact with the biological material associated with the electrical interconnects. To improve image clarity and improve alignment, an optical light source may illuminate the MEA to facilitate alignment via the optical detector.

“Cell culture parameter” refers to one or more physical parameters relevant for maintaining and/or growing a biological material. Generally, parameters relevant for cell culture include temperature, humidity, gas composition (e.g., CO₂ level in air), fluid media and composition thereof that is introduced to the biological material, pH, etc. In some applications, the biological material may be cultured cells. In other applications, the biological material may be tissue, including ex vivo tissue slices. The cell culture parameter is tailored to the biological material, including to promote cell growth and/or maintain biological material in a living condition (such as measured by a cell death and/or apoptosis assay), including over a time course that may span up to one day or more, such as up to a week, a month, or more.

An “optical interface” refers to electromagnetic radiation provided to the biological material and/or provided from the biological material. Such an optical interface can have a number of important functional benefits, including the ability to reliably align the PCB interface with the MEA, and thereby establish good electrical connection. Similarly, for applications such as optogenetics, fluorescent-activated imaging/actuation and the like, an optical interface broadens and multiplexes the application beyond just an electrical interface. Relevant components of an optical interface includes, but is not limited to, imagers, light sources, markers and probes, as well as calibrations and algorithms to implement alignment.

A “fluidic interface” refers to a fluid connection with the biological material, where fluid can be introduced to and removed from the region of biological material. Such introduction and removal can be useful to maintain biological material viability. Removal is also useful for collection of a biologically-produced or released material. Again, such an interface broadens and multiplexes the application beyond just an electrical interface. Relevant components of a fluidic interface includes, but is not limited to, pumps, valves, flow controllers, media, containers, tubing, connectors and the like.

In the following description, numerous specific details of the devices, device components and methods of the present invention are set forth in order to provide a thorough explanation of the precise nature of the invention. It will be apparent, however, to those of skill in the art that the invention can be practiced without these specific details.

The invention can be further understood by the following non-limiting examples.

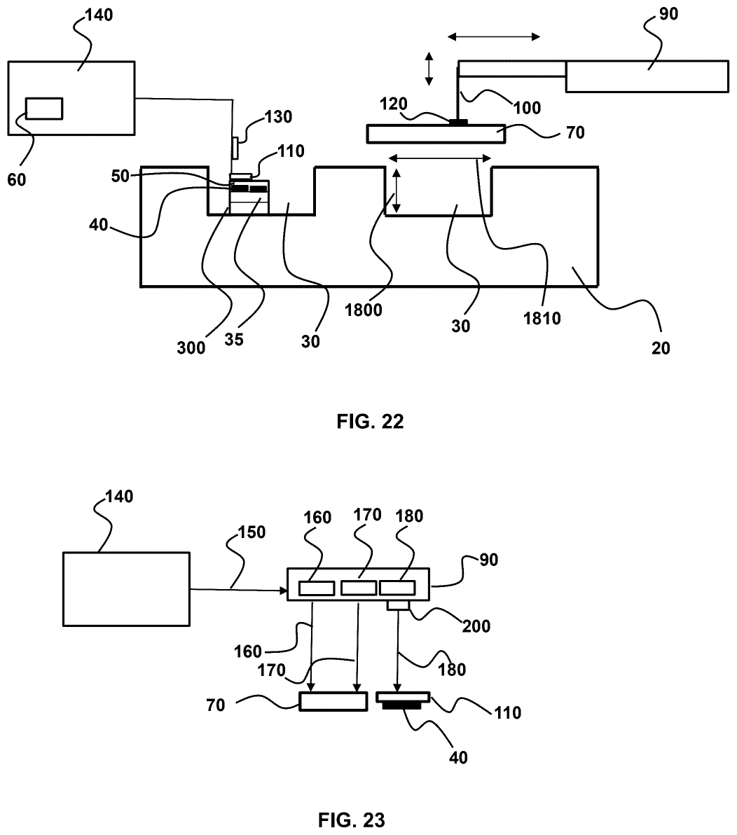

Referring to FIG. 22 (not to scale), in an embodiment an automated system has a sterile platform 20 comprising a plurality of recess features 30. The recess features may have a geometrical shape that is cylindrical, defined by a diameter 1810 and a depth 1800 selected to receive biological material 35 and MEA 40, including within a container 300. Electrical interconnects 50 provide an electrical interface with the biological material 35. The electrical interface permits measurement of one or more electrical parameters, including electric potential, at each electrode of the MEA, and a corresponding electrical parameter map of a biological material, such as neuronal cells. A downstream signal acquisition unit 60 measures and records electrical signal from biological material via electrical interconnect(s) 50. Lids 70 may cover the recess feature between interfacing events. For clarity, FIG. 22 schematically illustrates that lid 70 is removable and placeable by a robotic positioner 90, as illustrated by double sided arrows with the left side recess feature that is uncovered, and more specifically via an end effector 100. Of course, the controllers, electronics and the like may be incorporated into robotic positioner 90; for clarity, they are illustrated separately on the upper left side of FIG. 22 with another robotic positioner illustrated at the right side removing the lid 110. In other words, a single robotic positioner may incorporate both elements for lid removal and for electrical interfacing.

With respect to the electrical interfacing with biological material 35, a printed circuit board (PCB) 110 connected to the end effector 100 electrically connects to the MEA 40, including via electrical interconnects 50. To facilitate robotic alignment between the PCB 110 and the MEA 40, an optical detector 130 integrated with the robotic positioner, including at the end effector, may be used to optically position the PCB with the corresponding MEA to ensure good electrical contact with each electrode of the MEA.

A controller 140 may be utilized to implement an electrophysiological interface scheme 150 between the robotic positioner 90 and an MEA, as summarized in FIG. 23 (not to scale). The electrophysiological interface scheme may comprise various aspects, including transmission of: a position signal 160 for positioner to position the end effector and deploy the lid grip and engage a desired lid of a corresponding recess feature; a removal signal 170 to the end effector portion of the robotic positioner to remove the lid from the recess feature; and an interface signal 180 to the robotic positioner to align the PCB interface 110 with the MEA 40. An optical light source 200 may be connected to the end effector for illumination to thereby facilitate optical alignment by optical detector of the PCB interface and the underlying MEA.

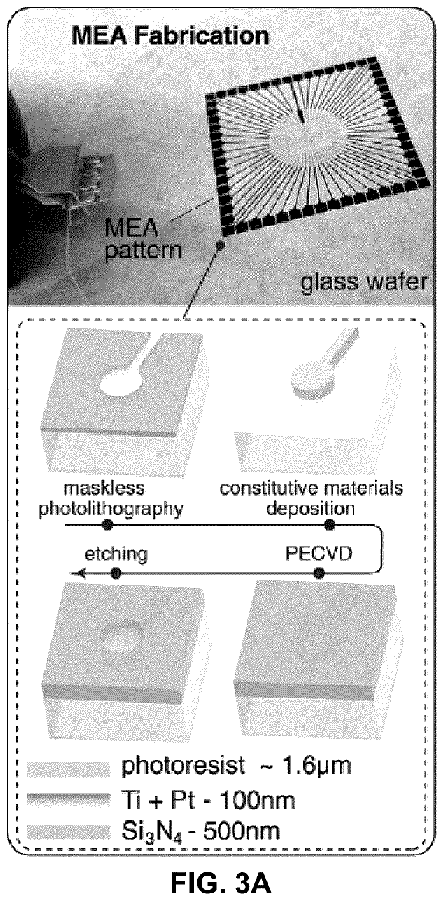

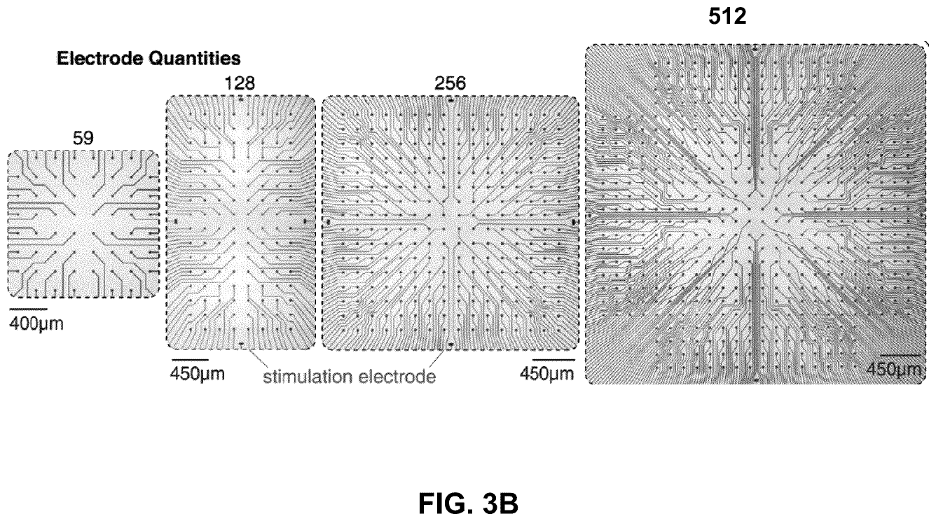

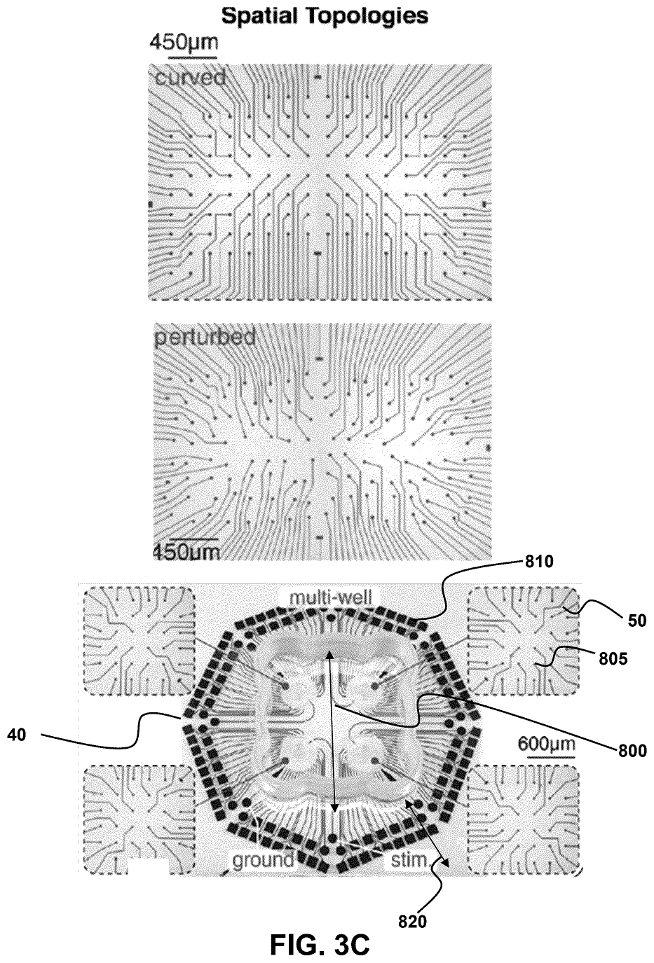

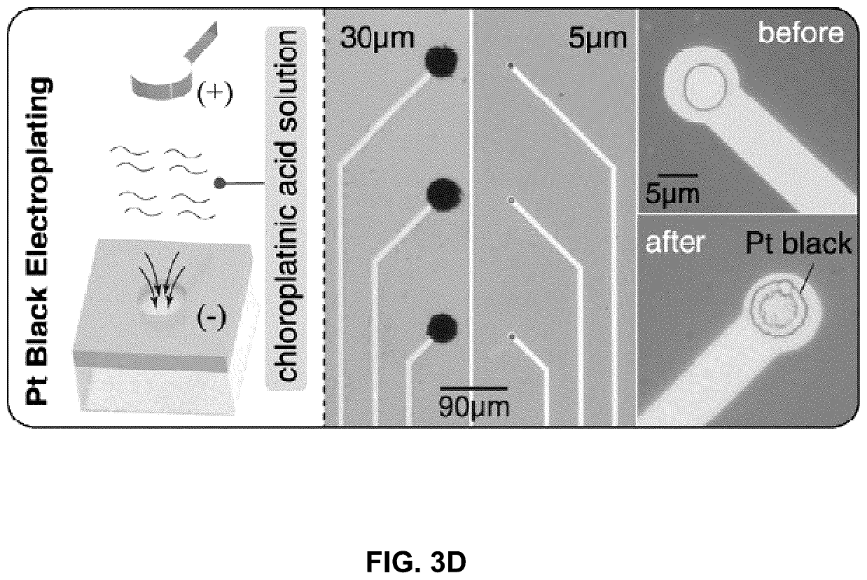

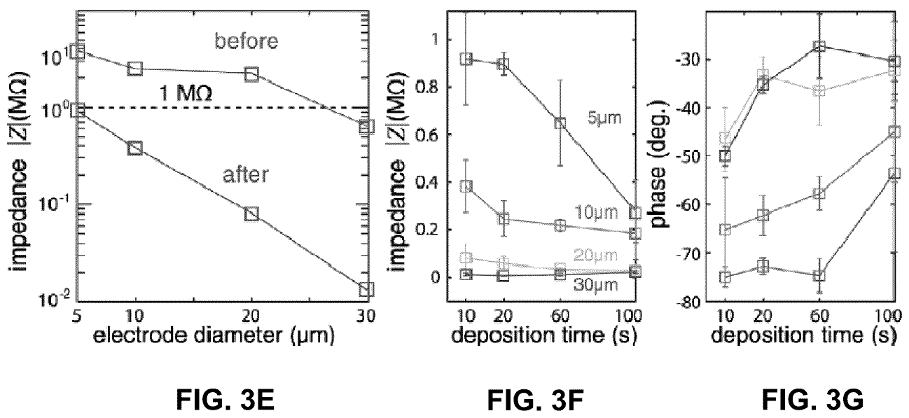

Referring to FIG. 3C, electrical interconnects 50 are provided on the MEA 40 and electrically connect (e.g., by physical contact) each microelectrode 805 positioned in a central region 800 (illustrated by 2-ended arrow). Plurality of contact pads 810 are positioned in an outer region perimeter orientation 820 (illustrated by the second 2-ended arrow), including for electrical contact with PCB interface. In this manner, the biological material can be positioned in electrical contact with MEA, particularly with contact pads and microelectrodes. The systems and methods provided herein are compatible with a range of geometries, as illustrated in the various MEA geometries, including number of electrodes, shape of the MEA (including a curved geometry), and for a multi-well configuration.

The controller may comprise various electronics, computer memory and connections (wired and/or wireless) as known in the art. For example, controller 140 may include computing device(s)) including one or more processors communicatively coupled to one or more memory devices (collectively referred to herein as downstream signal acquisition unit 60). Memory stores including, without limitation, by reading, writing, and/or deleting, data associated with operation of the automated system(s). In an example, memory includes a non-transitory computer readable medium which stores processor executable instructions encoded as software or firmware for implementing an electrophysiological interface scheme 150. When executed by the processor(s), the processor executable instructions cause the processor(s) to execute processor and memory operations that facilitate implementing the electrophysiological interface scheme 150 in an automated system, as shown and described above. In examples of an automated system where controller 140 is a mobile smart phone, memory thereof includes an app. In such embodiments, the app includes the non-transitory computer readable medium. In an example, computing device(s) implement and/or perform, at least in part, the functionality of sampling controller(s) in a system, either instead of, or in addition to, sampling controller(s) resident at or near location of the sterile platform, including as positioned in an incubator to support and maintain biological material.

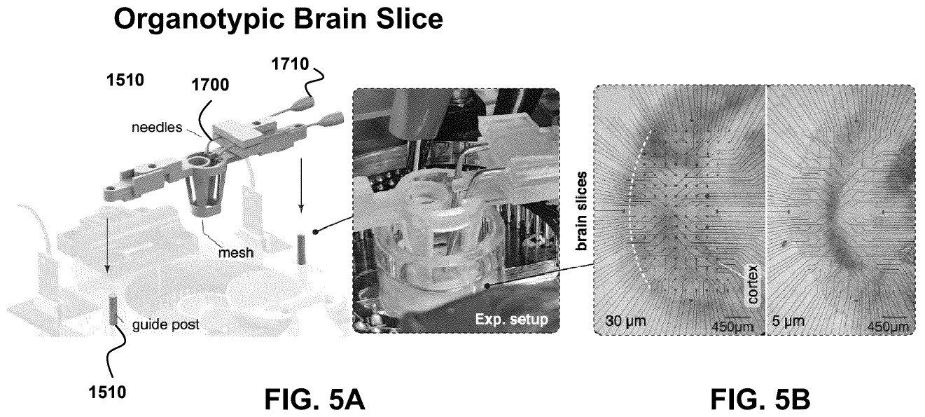

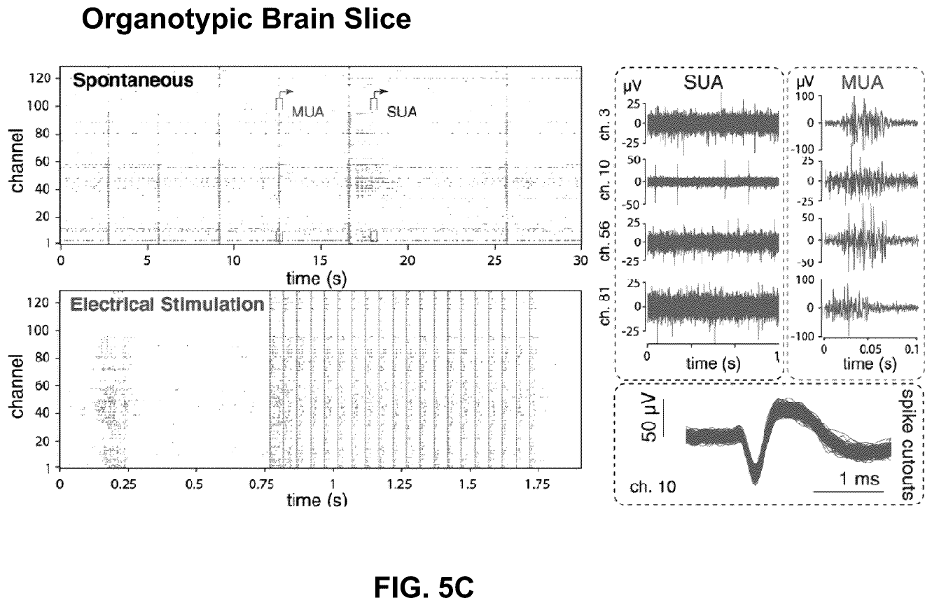

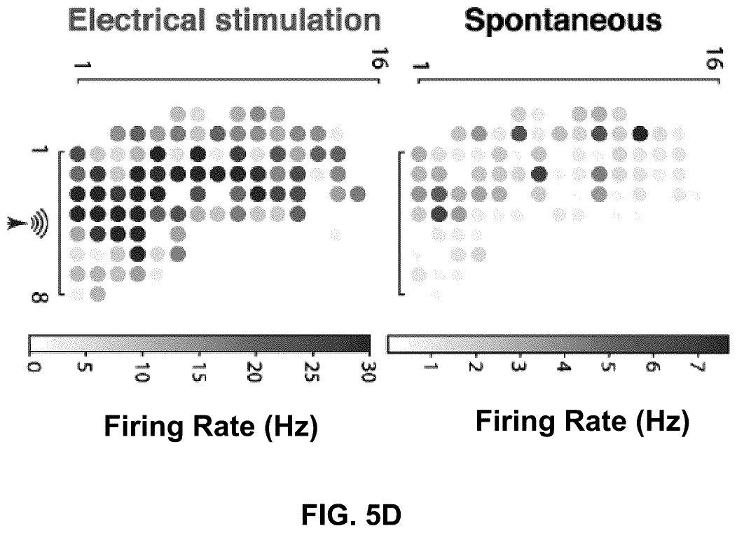

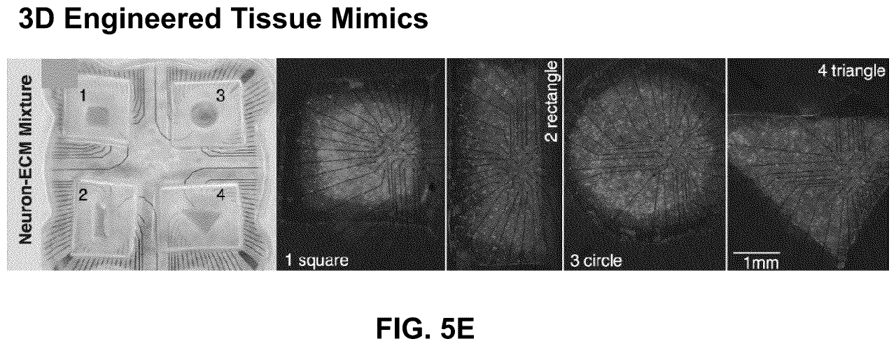

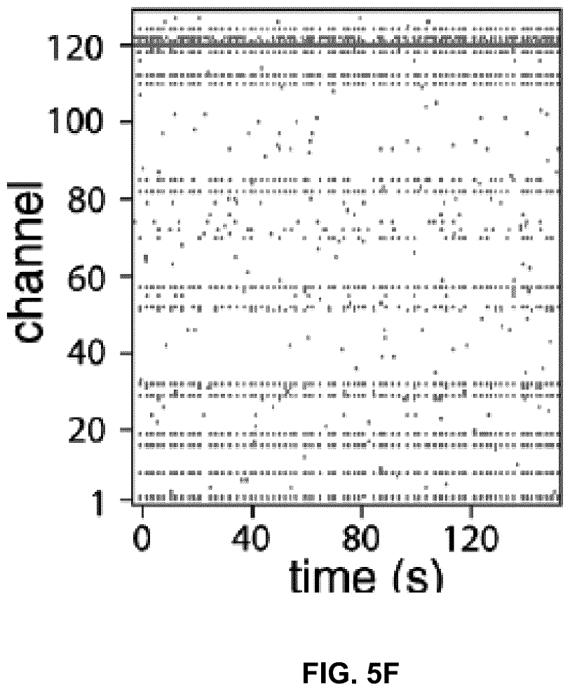

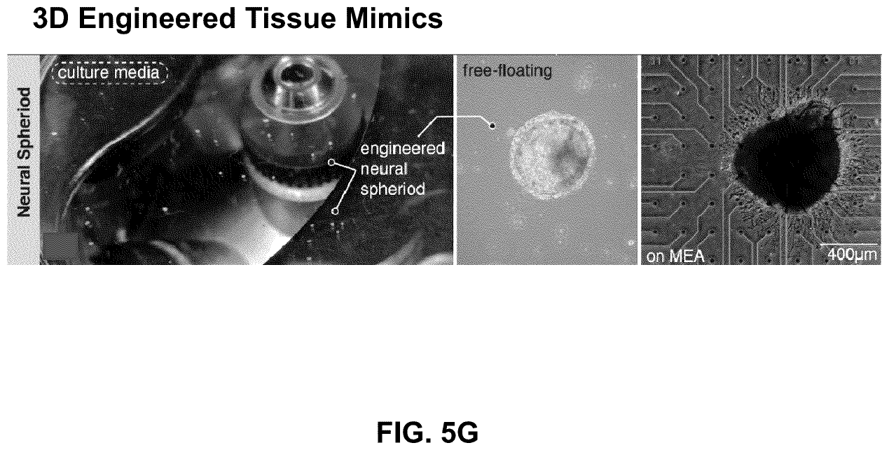

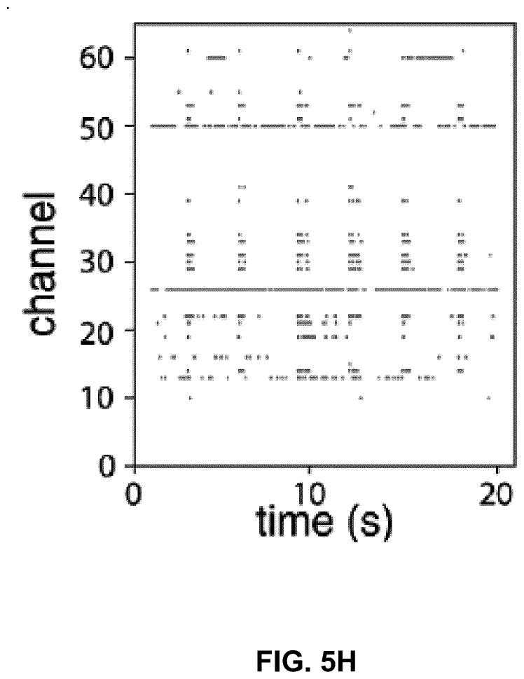

As discussed, there may be an optical interface (e.g., optical detector 130 such as camera and light source 200) to facilitate alignment. For support of biological material, there may be a fluidic interface 1510 (see, e.g., FIG. 5A). The fluidic interface may include an inlet tube 1700, and a source of cell media 1710, including to provide to recess features 30 in which biological material is positioned. An optical marker 1600, including a guide post, may help facilitate optical alignment. One advantage of an optical light source is that an optical probe that is optically activated can be activated by the light source.

Example 1: Mind In Vitro Platforms: Versatile, Scalable, Robust, and Open Solutions to Interfacing with Living Neurons

Neural tissue supports a host of information processes fundamental to many organisms, from autonomic body functions to motion, sensing, and high-level reasoning. [1-3] In the quest to decode the inner workings of neural architectures, their inspection has long relied on electrical recordings. [4,5] While in vivo electrophysiology, instrumental in neuroscience, uniquely allows for sampling neural activities associated with specific behaviors, [6,7] its interpretation is challenged by whole-organism complexity. In vitro systems, including single cells, small networks, or tissue samples of larger-scale connectivity, in contrast, represent a reduced, yet complementary, route to expose neural interactions across scales. [8-12] From the perspective of synthesizing fundamental principles of biological computing, there are significant opportunities for deploying electrical interfaces in vitro, particularly in conjunction with engineered neural substrates. [10-13] Indeed, by spatially distributing and connecting biological units (neural populations) of prescribed size, geometry, or neuron-type onto input/output electronic platforms, living processing architectures may be realized, operated, and tested, [14-20] enabling a new class of computing systems, with ramifications in engineering, biology and healthcare.

In this context, micro-electrode arrays (MEAs) technology, where electrodes of variable size, density, and spatial arrangement are patterned on biocompatible surfaces, represents a powerful and mature option to interface with cellular systems, [21] and has been employed from single neurons electrophysiology [22] to high-throughput pharmaceutical screens. [23-25] However, the potential of MEA systems remains hindered by design, fabrication, integration, and software management complexities. Labs have indeed little choice but to invest in commercial solutions, [26,27] which are proprietary, specialize to support biomedical research, afford minimal-to-no customization, and remain costly, limiting adoption (from tens of thousands of dollars for a standard 60-electrode system to hundreds of thousands for more advanced configurations). This has led to a recent interest in the open-source development of MEA platforms, [28,29] aimed at lowering barriers to entry while catering to needs such as multi-well drug testing, enhanced optical access, [31,32] or chronic tissue monitoring. [29] However, a comprehensive, accessible solution to flexibly and multimodally interact with a variety of neural substrates, from organotypic brain slices to engineered 3D tissue, remains to be demonstrated.

Here prompted by the untapped potential of in vitro neural systems for computing, we present an interfacing platform that is versatile, scalable, reconfigurable, and portable, that can be easily fabricated, robustly operated across cellular contexts, and broadly disseminated. We term this technology “mind in vitro” (MiV), to emphasize the information processing motivation. Our platform hosts MEA chips entailing up to 512 electrodes, manufactured in standard cleanroom facilities via maskless photolithography and varying in size, spatial topology and transparency depending on the application. These chips are matched to swappable, custom printed circuit boards (PCBs) relaying neural signals to Open Ephys [33] or Intan terminals [34] for signal acquisition and subsequent downstream processing. Our system seamlessly integrates with both electrical and optical stimulation modules, as well as other add-ons such as fluidic interfaces or tissue-specific positioning apparatuses. Reconfigurability is further leveraged to comply with industrial standards and integrate with common microscopic chambers, enabling concurrent imaging. Such flexibility allows for the combination of high-temporal (electric) and high-spatial (imaging) resolutions, seizing on the opportunities afforded by ever-expanding genetic optical markers used in neuroscience research. [35,36] Additionally, an open-source software package is developed to manage the system and support operability, data storage, analysis, and visualization. To increase usability, the source code is based on the Python language and native interfaces with external neurophysiology (NeuralEnsemble [37]) and machine learning (scikit-learn [38]) software suites are provided.

Our integrated systems are demonstrated across a broad range of in vitro settings, from 2D cultures of embryonic stem cell-derived neurons and dissociated hippocampal cells to organotypic brain slices and 3D engineered neural tissue mimics. Multiple applications are illustrated, including electrical, optical, and fluidic manipulation, concurrent calcium imaging, and long-term recording (>24 h). By logging over 1000 h of experiments and tens of terabyte (TB) of data across distinct labs, robustness and portability are further showcased. By open-sourcing all design files, preparation protocols, documentation, and software, a useful, accessible, and self-contained neural interfacing solution is then delivered, catering to the expanding needs of both traditional and non-traditional in vitro applications.

### Platform Overview

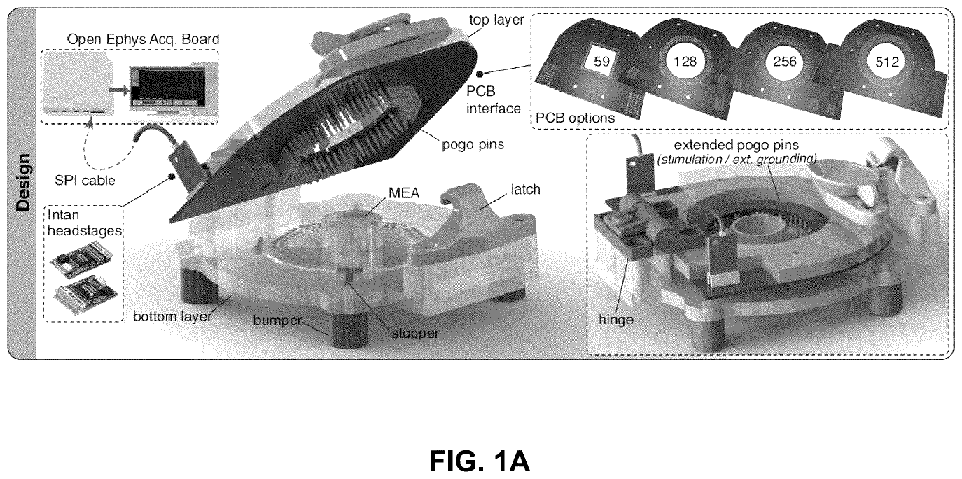

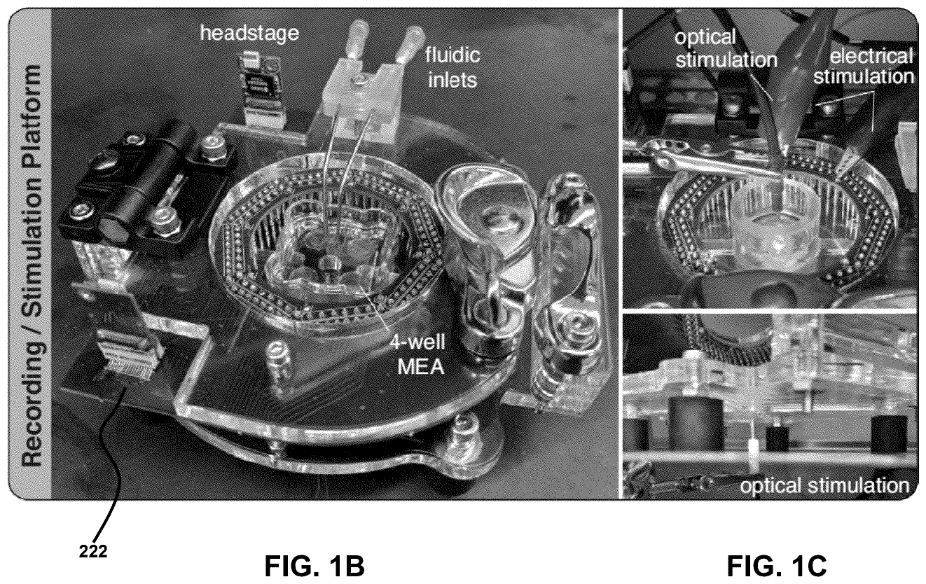

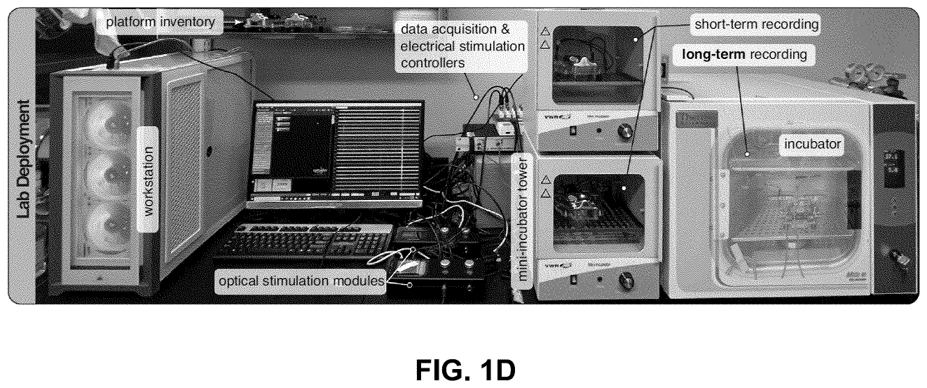

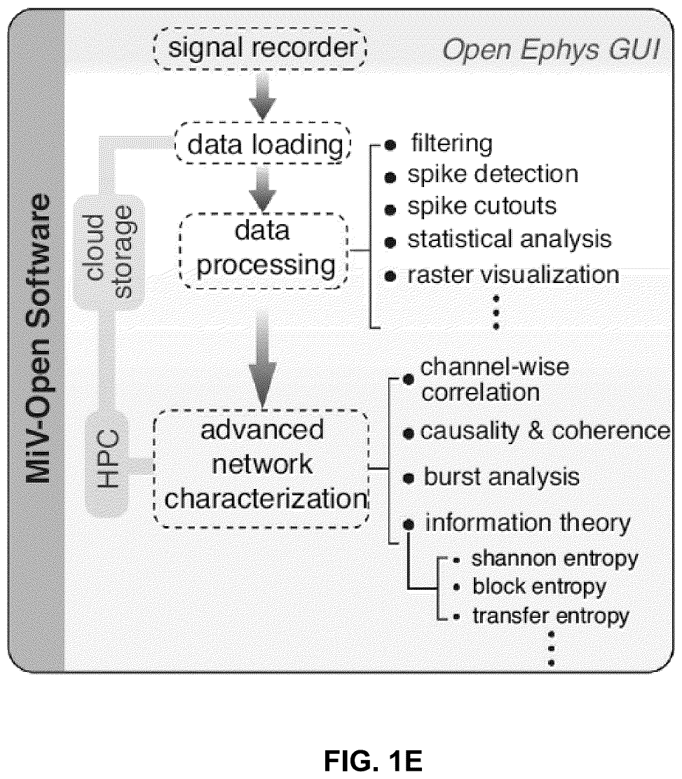

Here, a system-level overview of our hardware and software is provided (FIG. 1A-1E). We start by describing the recording platform, its basic configurations and extensions. We then discuss its deployment in the lab and managing software. Finally, the instant design is compared to existing alternatives.

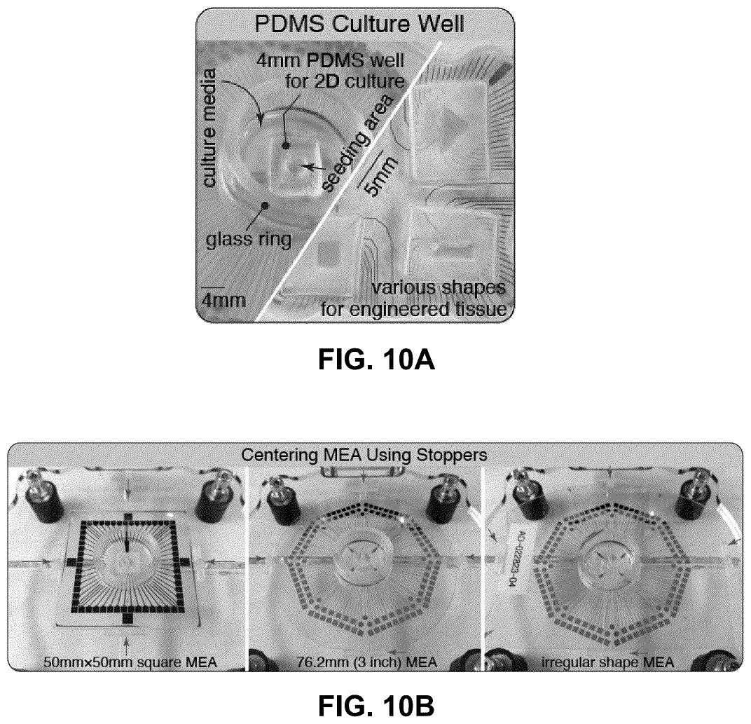

Recording Platform: Central to our system is a platform that hosts MEA-chips and PCB interfaces to form a complete signal chain, from neural substrates to data acquisition terminal and downstream processing (FIG. 1A). The platform comprises two layers of laser-cut acrylic board that are connected by a plastic hinge and can be locked in-place through a latch. This design eases the loading and un-loading of the chips and allows for rapid changes in recording configurations. The bottom layer also accommodates four sliding guides and 3D-printed stoppers to conveniently center and hold chips of different sizes and shapes (FIG. 10A-10B). Underneath the bottom layer, four soft rubber bumpers are affixed to insulate the platform against vibrations, minimizing recording noise. Secured to the top acrylic layer, a PCB provides the signal interface between the MEA chip and the Open Ephys data acquisition terminal. To ensure a firm yet compliant contact with the MEA, arrays of spring-loaded contact (pogo) pins are soldered to the bottom of the PCB. Upon loading a chip, the top layer is closed and locked, with the pins gently making contact with metal pads patterned on the perimeter of the MEA. These pads and the electronic tracks emanating from them are designed to connect one-to-one with micro-electrodes located at the center of the chip, where neural substrates are plated and incubated. Signals sampled at 30 kHz at each electrode are then transferred through these tracks, pogo pins, and PCB before being received and processed by amplifier headstages located on the rear edge of the PCB. Intan Technologies headstages, connected to Open Ephys terminals through serial peripheral interface (SPI) cables, are utilized here because of their wide compatibility.

Supported Recording Capacities: With versatility in mind, the instant system supports a range of recording capacities and configurations. The Open Ephys board allows the connection of up to 4 SPI cables, each capable of handling up to 128 digital data streams, setting the maximum capacity to 512 recording channels. This translates into MEA chips of 512 micro-electrodes. Practically, in certain contexts, it may be desirable to employ fewer recording channels. Indeed, fewer channels imply fewer headstages (reducing costs), simpler MEAs and PCB designs, as well as less computationally intensive data processing and analysis. Our system then provides built-in modularity to mix-and-match Intan headstages of variable capacity (32, 64, and 128 channels), through a series of swappable PCB options sup-porting 59 (compatible with the widely used multichannel system 60MEA series), 128, 256, and 512 recording channels. An example of a 128-channel recording system employing two 64-channel headstages, is illustrated in FIG. 1B, including with swappable PCB 222. Assembly details for each standard are found in Table 2.

Extension to Electrical and Optical Stimulation: The system supports multiple interfaces, including both electrical and optical neural (biological material) stimulation, thereby providing the ability to control and introduce multimodal inputs to the neural (biological) tissue. All electrodes on the MEAs are bidirectional and can be used for high temporal resolution and multi-frequency electrical stimulation by connecting them to an external stimulator. Stimulation tests presented below demonstrate the use of a low-cost stimulator with two independent output channels (Stimjim [39]), which can be directly connected with the electrodes through extended pogo pins on the PCB (FIGS. 1A and 1C). Alternatively, if a larger number of stimulation channels is desired for fine spatial control, a bidirectional Intan controller can be connected to the platform's headstages, enabling the simultaneous stimulation of any combination of up to 128 electrodes on the MEA (details in Table 3). Spatiotemporal stimulation patterns and sequences can be prescribed, saved, and loaded as configuration files through an electrophysiological interface scheme software, including, for example, via software (see below). Another powerful, widely adopted modality of neural interaction is optical stimulation. At the cellular level, the expression of light sensitivity to specific wavelengths is achieved via channel-rhodopsin transfection. [40] Here, we employ 465 nm LEDs and lasers from Doric lenses connected to optic fibers to locally stimulate transfected neural populations. To this end, our platform is designed to be top and bottom accessible, with openings on both acrylic layers, allowing optical fibers to be placed underneath or above the MEA-chip (FIG. 1C). This design facilitates flexibly combination of recording and electrical/optical stimulation, either concurrently or serially, to achieve the desired input/output protocol.

Deployment in the Lab: The platform deployment is presented in FIG. 1D, illustrating its integration with a workstation, multiple data acquisition/stimulation controllers, optical/electrical stimulators, and incubators of different dimensions and specifications, all fitting within the space of a standard benchtop. With the largest dimension below 150 mm, the recording platform, upon sanitization, can be easily placed and operated across incubators. Apart from providing the necessary environment for maintaining biological cultures, including neural cultures, incubators also serve as noise canceling Faraday cages that minimize interference, thanks to their conductive inner surfaces. Accordingly, any of the systems provided herein may positioned the biological material, with attendant platform that supports the biological material, within an incubator that also functionally serves as a Faraday cage to minimize electrical interference. Assembled from inexpensive materials, the recording platform can be conveniently duplicated to allow parallel recordings. Short-term experiments (<2 h), during which culture media evaporation and pH change are not a major concern (at least in the case of dissociated cell cultures), are performed in a mini-incubator tower stack. Maintaining only temperature, these incubators are low-cost, portable, and have simple chambers that can be easily reconfigured and sanitized. Several demonstrations have been carried out in this environment. For long-term recordings (>24 h) instead, more capable incubators for the control of temperature, pH, and humidity are used. The required high-humidity levels, in particular, pose a significant challenge for electronics, leading to rapid oxidation and signal degradation. This is one reason why long-term in vitro electrophysiology remains uncommon. In our case, we adopt a system configuration where the data acquisition terminal is physically separated from the on-site signal amplification so that the recording platform can be placed inside an incubator for long-term experimentation. [41] This, in combination with the use of inexpensive components, reduces the liability of operating our platform in incubator atmospheres for extensive periods of time, minimizing financial losses in the event of electronics failure. A 24-h experiment with no appreciable hardware degradation is reported.

Software: Handling large streams of electrophysiology data, particularly when a high number of recording channels or multiple platforms are used in parallel, introduces challenges in compressing/archiving, transferring, and processing collected information. For reference, a single 24-h experiment carried out with a 512-channel platform sampling at 30 kHz produces ˜4 TB of data. In light of this rising demand, presented is a cloud computing solution that simplifies the management of terabyte-size data and streamlines post processing. The software offers a user-friendly interface for experimentalists who desire to construct processing pipelines and view results on their local desktop, while power-users can utilize the backend tools that support scalable high-performance computing (HPC) for custom, large-scale analysis. A software overview is presented in FIG. 1E.

The core of our software is a Pythonic pipelining platform specifically developed for maximizing flexibility and promoting consistency in experimental research. Our software provides a structured analysis template, while its backend incorporates essential features that are frequently used for analysis, eliminating the need for user implementation or maintenance. These features include caching, data pipelining, filtering, [43-45] spike detection, [46,47] principal component analysis (PCA), [48] burst detection, criticality analysis, [49,50] as well as an array of visualization functions. [51,52] In addition, the software includes HPC support to integrate existing algorithms and pipelines into supercomputing clusters, [53] enabling parallel processing, and I/O capabilities [54] to accelerate large-scale analysis. [55,56] This template relies on common data structures and order of operation, ensuring compatibility among different data sources, methods, and algorithms. Furthermore, this approach modularizes the software, where users can selectively install only needed components as plug-ins, resulting in a compact and lightweight package.

To maximize impact, our software natively integrates with a variety of external packages of demonstrated utility, such as H5py with standard H5-data structure for scalable I/O, [57] Aim UI for slurm monitoring, Jupyter server for interactive graphical user interface (GUI), PyInform/IDTxl for multi-variate analysis, [59,60] Globus APIs for synchronizing data stream, NeuralEnsemble [61,62] or Kilosort [63,64] for neurophysiology processing, and scikit-learn for machine learning. Besides enabling the effective use of our platform, our software ecosystem aims at bridging the gap between experimental practice, data management and advanced analysis. While an in-depth characterization and demonstration of this software is beyond the scope of this paper, we note that all case studies presented here have been configured, analyzed, and visualized using it.

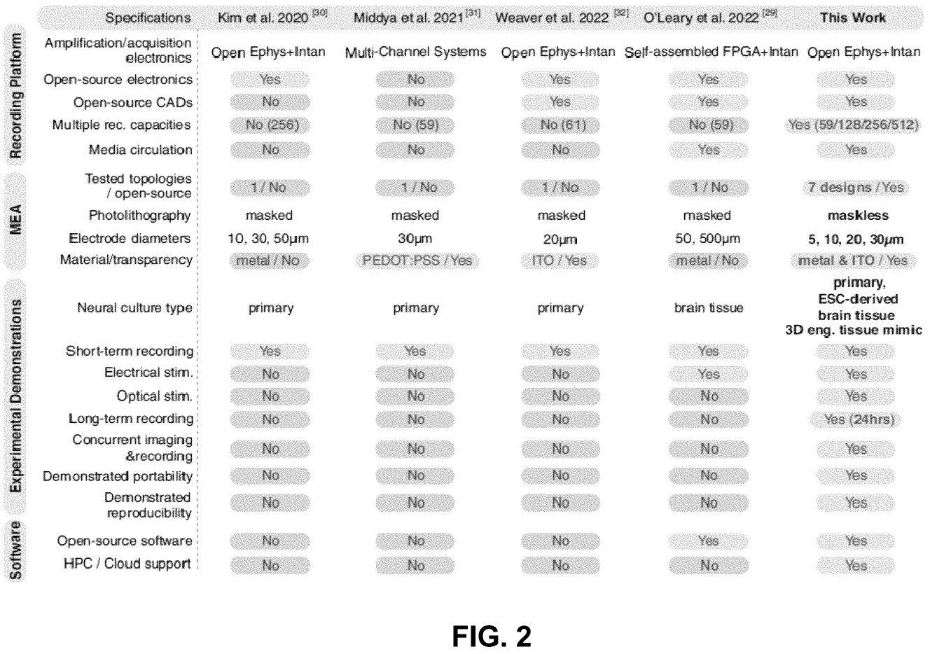

Comparison with Alternatives and Cost Breakdown: For the major specifications illustrated, we summarize and compare our in vitro electrophysiology approach with other previously reported custom solutions. As illustrated in FIG. 2, from a recording system perspective, our approach demonstrates the highest level of versatility while being fully committed to open-sourcing both hardware and software. Further, and importantly, the robustness and utility of our system are comprehensively demonstrated across a range of neural substrates and stimulation settings, including long-term and off-site recordings, as well as through extensions such as fluidic circulation and concurrent imaging.

A cost estimate is also provided. Incorporating open-source components as well as inexpensive, accessible materials, our platforms can be assembled at a fraction of the investment necessary for a comparable commercial product. Indeed, our platforms range from ˜$2500 for a 59-channel system to ˜$12,000 for a 512-channel device (both including the Open Ephys terminal), delivering ˜10-25× cost reductions, depending on vendor and comparative specifications. Given the modularity of our approach, this price advantage becomes even more significant considering that a single fabricated system can be reconfigured to satisfy a number of different or evolving needs. Details of the cost breakdown can be found in Table 2.

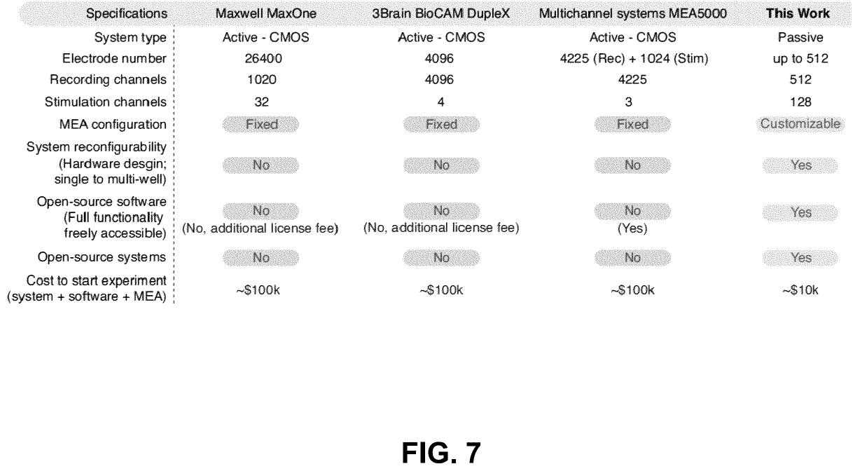

We further extend our comparison to complementary metal-oxide-semiconductor (CMOS)-based commercial high-density MEAs (HD-MEAs). [65] Incorporating thousands of densely packed electrodes, those systems provide high-resolution detection of single-cell and population-level activity. However, those solutions are closed-source and monolithic, limited in terms of independent electric stimulation channels and available configurations, and they are costly (upward of ˜$100k). Further, CMOS chips are difficult to manufacture, requiring high-tech facilities and thus preventing in-house sensors' customization or replacement. This is one main reason why we chose a non-CMOS technology for our devices. A detailed comparison between our systems and leading HD-MEA products can be found in FIG. 7.

### MEA-Chip Microfabrication, Design, and Characterization

In keeping with an approach centered on versatility, here we focus on the realization of custom MEA-chips. We adopt a microfabrication process based on maskless photolithography. This technique enables the direct patterning of any 2D topology imported from CAD files, bypassing the need for dedicated photomasks for each design. Chips can then be customized without additional manufacturing steps, reducing fabrication time and cost. Further, this protocol allows significant flexibility in terms of constitutive materials, which we demonstrate and leverage to manufacture transparent MEAs for optical applications, as described below.

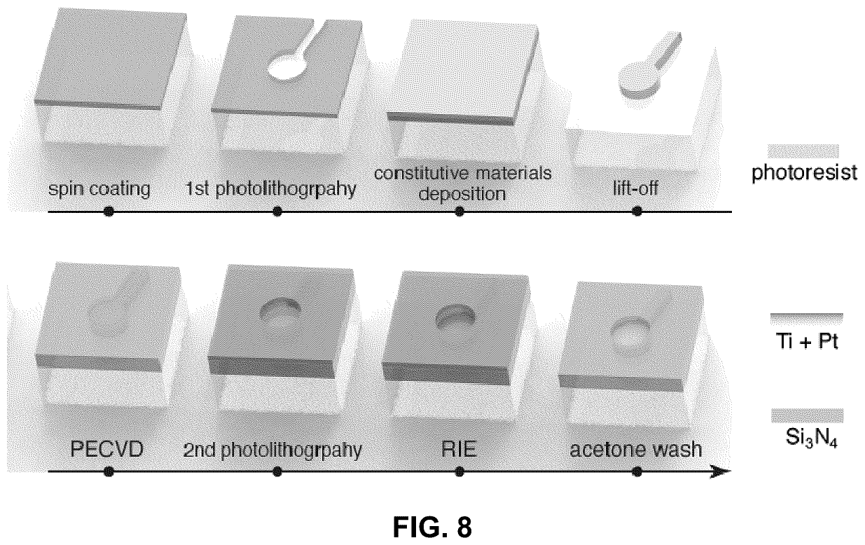

Microfabrication: An overview of the adopted fabrication process is illustrated in FIG. 3A, while detailed information about tools and settings is provided below and FIG. 8. We start by spin-coating a thin layer of photoresist (PR) onto a clean borosilicate glass wafer. Glass is selected as a substrate material because of its transparency, chemical stability, and strength. We employ the Heidelberg MLA 150 Maskless Aligner to pattern custom MEA designs on the PR layer via a direct-writing laser source. The processed wafer is then submerged in developer solution to dissolve the exposed PR and reveal the MEA topology. Next, the constitutive materials of the MEA are uniformly deposited on the developed wafer through sputtering. Most of the MEAs employed in this study consist of two metal layers of titanium (Ti) and platinum (Pt), with a total thickness of 100 nm. Here, Pt serves as the main constitutive material because of its good conductivity and relative ease to obtain and utilize, while the Ti layer is used for enhancing adhesion between Pt and wafer. An alternative process is also discussed below for the deposition of thin films of indium tin oxide (ITO), which has been previously demonstrated in fully transparent MEAs. [32] Subsequent to the sputtering process, materials outside of the MEA pattern are lifted-off through sonication in acetone.

Next, we apply the passivation layer that encapsulates the entire MEA, with the exception of the electrodes and contact pads. To do this, the wafer is first deposited with a 500 nm thick layer of silicon nitride (Si₃N₄), through plasma-enhanced chemical vapor deposition (PECVD). Then, a second photolithography process is performed, creating a patterned PR layer on top of the Si₃N₄, with openings at the electrode and contact pad locations. The exposed Si₃N₄ is then removed by reactive-ion etching, using the PR layer as a mask. The PR layer is subsequently stripped using acetone. The overall result is a chip in which electrodes and contact pads are exposed for recording and stimulation while connecting wires are insulated. Finally, the wafer is diced into chips of desired shapes.

Design: In FIGS. 3B and 3C, we demonstrate the versatility of the above approach through the realization of a variety of MEA designs. FIG. 3B illustrates a series of 30 μm-electrode chips of increasing resolution spanning 59 to 512 channels arranged in regular rectangular patterns. Each of these MEAs presents a contact pad layout compatible with one of the PCB interfaces in FIG. 1A, synergistically enabling the rapid reconfiguration of the platform. Besides the number of electrodes, the chip layout can be modified as well. FIG. 3C illustrates three designs, of which the first two have not been previously reported (either commercially or through customization): a curved MEA arrangement optimized to conform to the natural morphology of the mouse cortex, and a perturbed layout in which electrodes are randomly displaced around uniform grid locations to reduce subsampling and aliasing in the analysis of neuronal avalanches [9] as theoretically predicted in ref. [68]. The third example implements a multi-well topology, allowing it to host and monitor four independent cultures at once for parallel testing. The use of several of these layouts is demonstrated below.

Characterization: Prior to deployment for cell seeding, electrical properties of the chip arrays are characterized, and their impedance is reduced through platinum black deposition. This is an electroplating technique for which platinum in chloroplatinic acid solution forms granular structures that adhere to the electrodes upon current application [69,70] (FIG. 3D, further details below). The process increases the electrodes' effective surface area, lowering impedance and thus improving their capability of detecting neuronal signals.

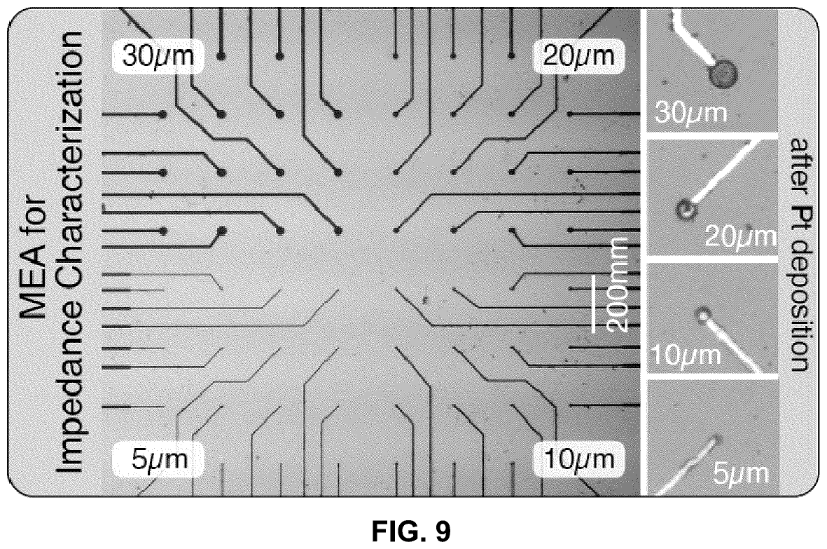

In order to gain operational insight, we characterize the dependence between impedance and electrode size. We employ a specifically designed chip (again leveraging the versatility of our fabrication approach) in which electrodes of four distinct diameters (5, 10, 20, and 30 μm) are patterned in separate quadrants (FIG. 9). FIG. 3E shows that, while impedance is generally larger for smaller electrodes (as expected), in all cases, the use of platinum deposition leads to reductions of at least one order of magnitude, yielding values below the neural signal detection threshold of 1 MQ. This implies that all of these electrode sizes are viable, an additional asset relevant in specific applications. We also explore how impedance decreases relative to deposition time (FIG. 3F). Our results suggest that longer deposition times (of up to 100 s) are needed for smaller electrodes (5-10 μm) to achieve the ideal value of ˜200 kΩ. [71] Finally, we quantify the phase of the impedance (which is a complex variable) to confirm its negative value, that is, to confirm the capacitive property of our electrodes. As can be seen in FIG. 3G, this is demonstrated across all cases, rendering our chips well-suited for electrophysiology. By continuously monitoring the chips' impedance throughout their lifespan, we observe sustained chip functionality over approximately three-month periods of heavy-duty utilization (back-to-back experiments, electric stimulation, detergent, and plasma cleaning), before deterioration. While this work focuses on neuron types and characterizations that require relatively shorter chip usage windows (˜1 month), it is important to highlight that robust performance over longer timeframes allows for culturing cells that demand extended preparation times (>1 month), such as human induced pluripotent stem cells-derived neurons. Furthermore, the systems provided herein are not limited to neuronal cells, but are applicable to any biological material where an electrophysiological interface is desired. Generally, the biological material may be referred herein as an electroactive biological material, as the biological material may undergo a change upon exposure to one or more electrical parameters and/or may itself generate a change in one or more electrical parameters, including an electric potential, electric potential field, a current, an action potential, or the like.

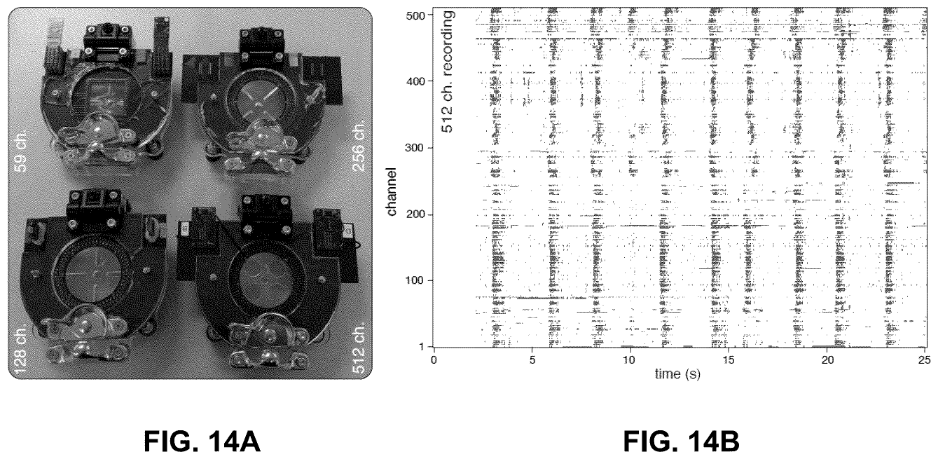

RESULTS: Through a variety of 2D and 3D neural systems derived from a broad number of sources, we illustrate the functionalities of the disclosed hardware and software, both on-site and off-site, involving multi-modal stimulation, concurrent imaging, and fluidic support. All demonstrations entail the use of a 128-channel platform configuration, selected because of its exact compatibility with both Open Ephys and Intan bidirectional modules. However, results relative to our other configurations can be found in FIGS. 14A-14B.

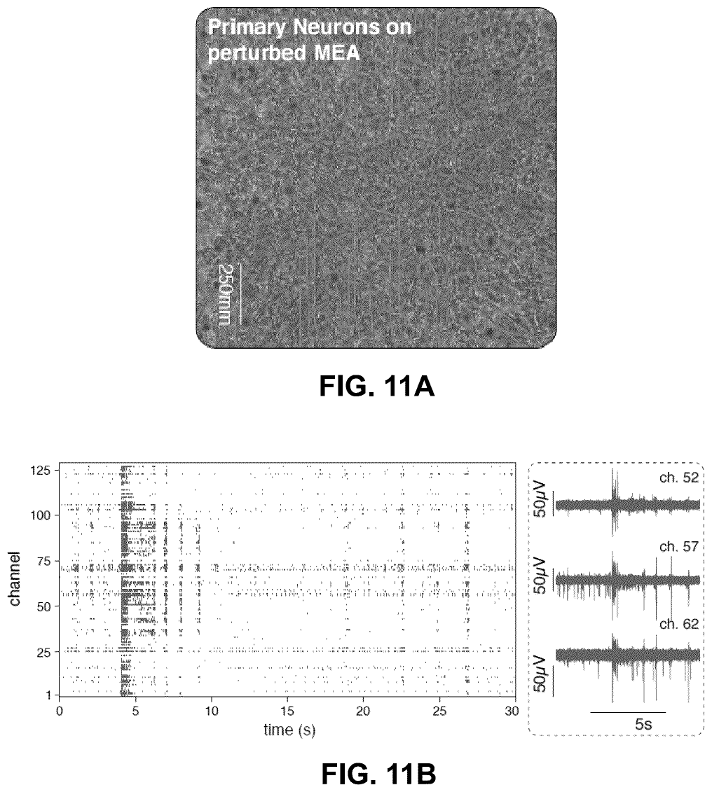

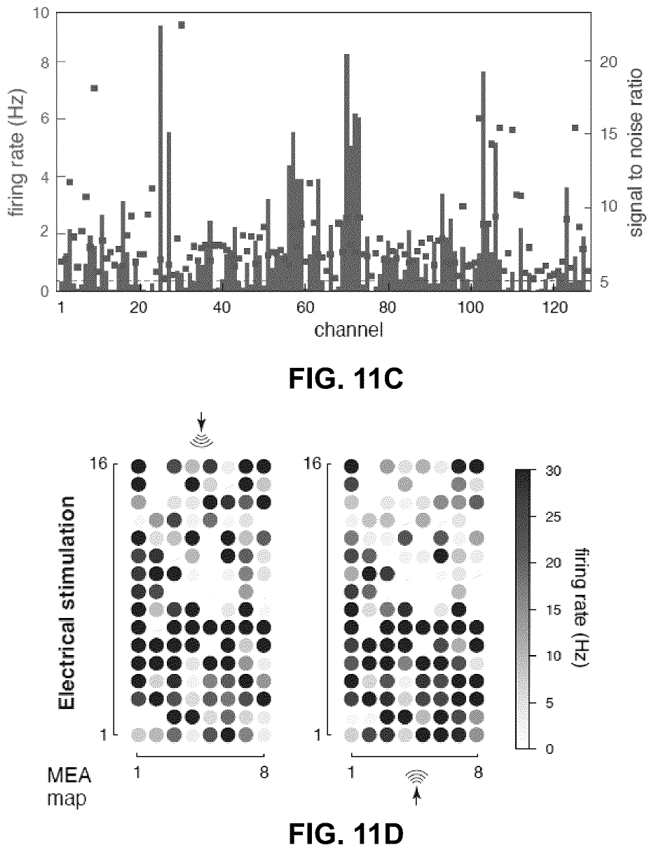

2D Neural Systems: We first test our interface via 2D cultures from both mouse embryonic stem cell-derived motor neurons (ESC-MNs) and dissociated mouse primary neurons (PNs). These cultures are prepared following different dissociation/differentiation protocols but share a conserved seeding procedure. Seeding is directly performed on MEAs that are surface treated to enhance cell attachment (protocol details below). Throughout, ESC-MNs represent our main cell line, as it endogenously expresses Channelrhodopsin-2 (ChR2) enabling optical stimulation, and because it can be differentiated into motor neurons that co-express enhanced green fluorescent protein (eGFP). This conveniently allows us to monitor culture conditions during preparation and maturation. Further, recorded data can be more easily interpreted with the aid of fluorescent microscopy. Results in this section are then shown for ESC-MNs. However, a parallel study with PN cultures (without GFP or optogenetic stimulation) is reported in FIGS. 11A-11D.

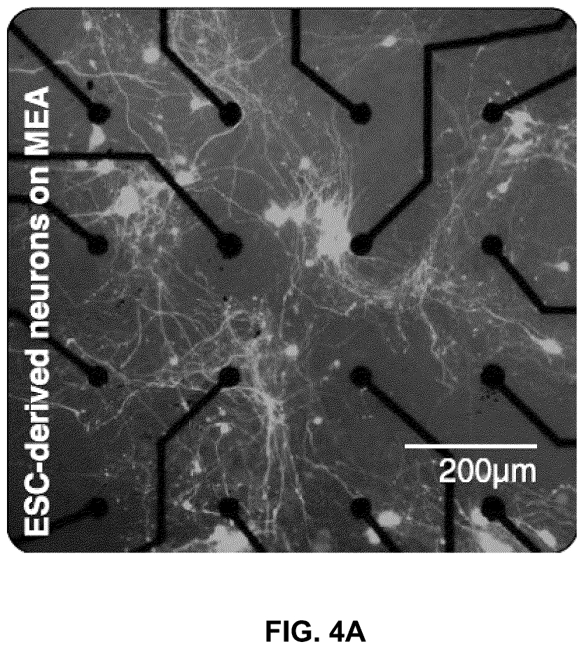

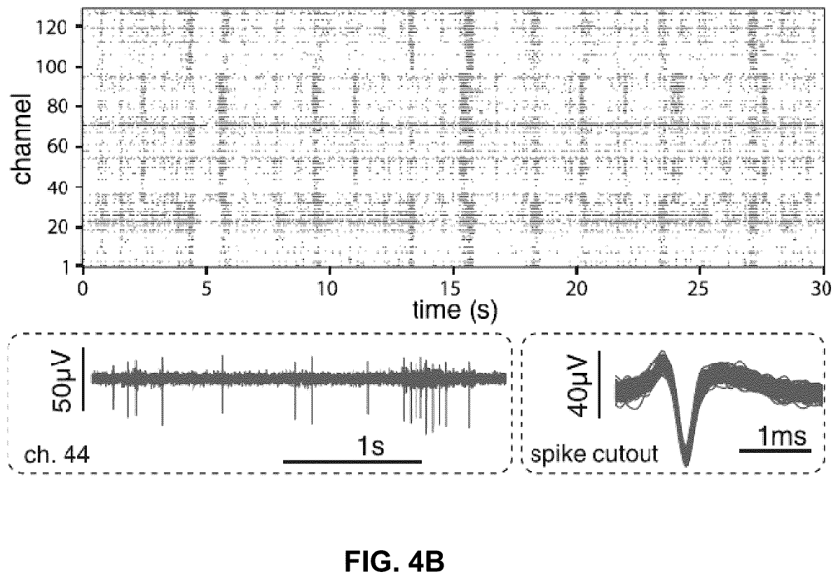

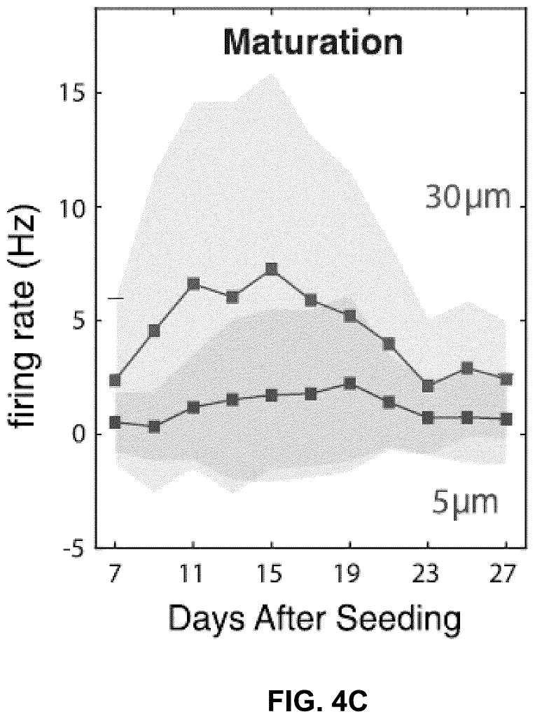

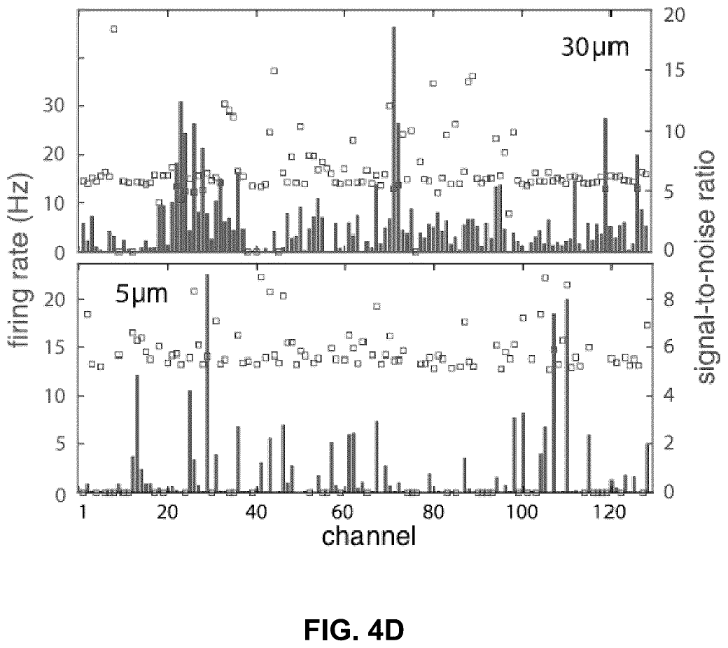

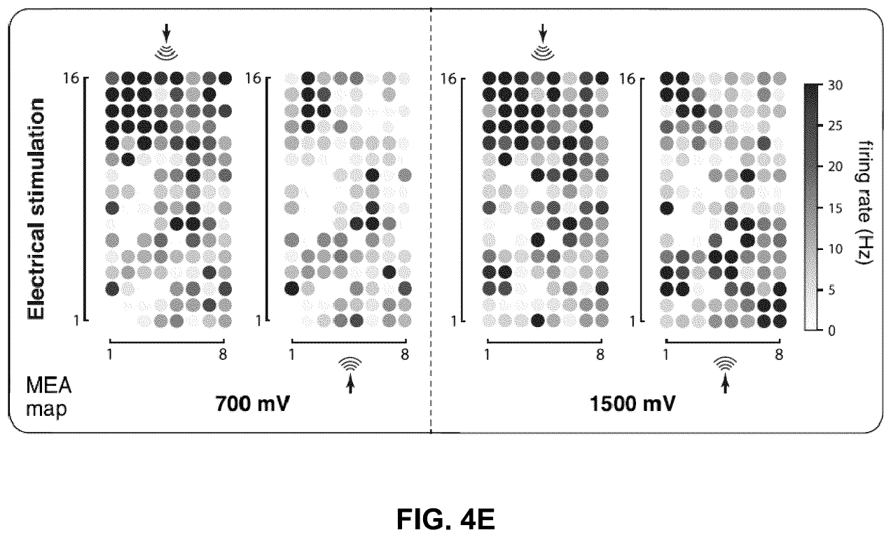

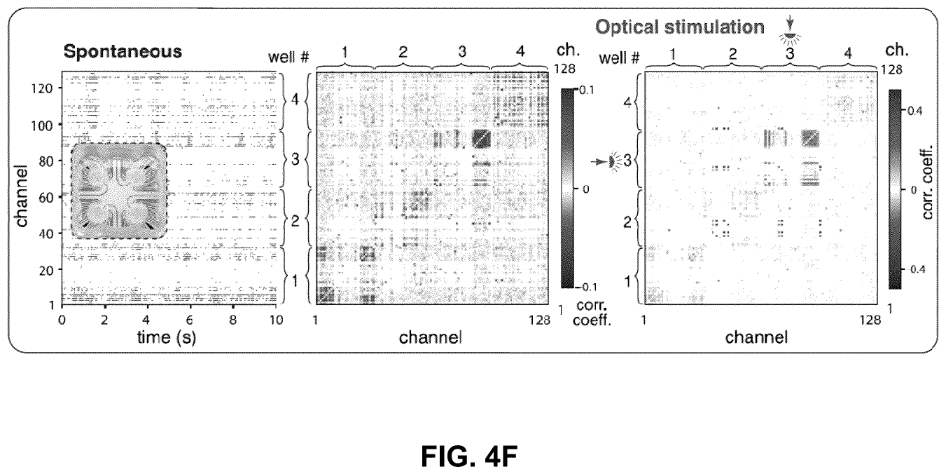

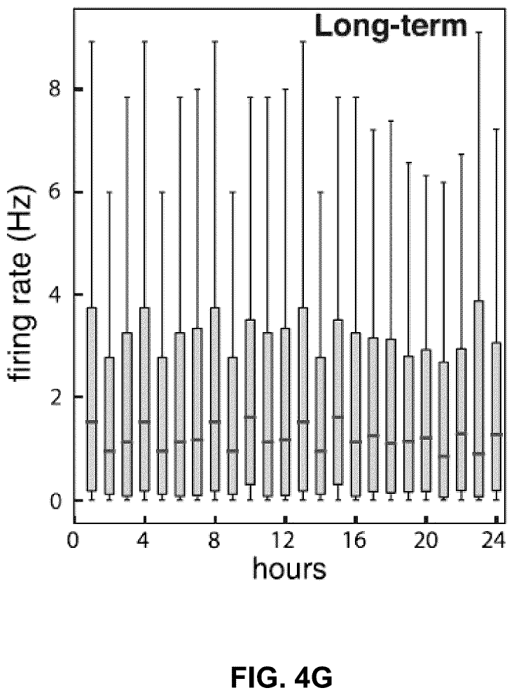

We seed ESC-MNs on two 128-channel MEAs, realized with either 30 or 5 μm electrode diameters (FIG. 4A), and begin recording spontaneous neural activity 7 days after seeding. Each recording comprises 2 min of data and is repeated every other day, at the same time, for 20 consecutive days. Raw neural signals are recorded using Open Ephys and are then processed offline through our software, for filtering and spike detection. The spike raster in FIG. 4B showcases spontaneous neural activity from the 30 μm MEA, where single unit activity is plotted across channels. The use of MEAs helps reveal intrinsic network properties, reflected here in highly synchronized population bursts, which would not be detectable at the single site level (e.g., patch clamp electrophysiology). Waveforms from all recording channels are sorted using PCA to isolate signal fingerprints matching characteristic neuron spike shapes (cutouts of FIG. 4B). The performance stability of our system is illustrated by plotting daily average spiking rates across all channels over 4 weeks, where a typical in vitro neuron maturation-degradation curve is observed. As can be seen in FIG. 4C, for MEAs of both electrode sizes, average spiking rates first increase during network maturation, to then plateau and gradually decrease as cells begin to age. This trend, including the timeframe of peak activity, is consistent with the trend reported in ref. [13], where recordings for the same cell type were performed using a commercial system. We note that the difference in firing rate amplitudes in FIG. 4C is not necessarily surprising, since larger electrodes can detect signals from a broader spatial range. We also quantify the quality of our recording setup by determining the mean signal-to-noise ratio (SNR) across channels. As seen in FIG. 4D, spiking channels (95% of channels for 30 μm MEA and 70% for 5 μm MEA) consistently exhibit an SNR >5 after filtering, on-par with reported performance of commercially available devices. [75]

On top of spontaneous activity recording, we consider simultaneous multi-modal stimulation. While this is a useful paradigm in neuroscience for studying synaptic potentiation, it is also a prerequisite to encode inputs for possible computing applications. As an example of electrical stimulation, we consider a single-well MEA seeded with ESC-MNs, and apply brief, biphasic electric pulses (Stimjim [39]) at a designated location. The heat-maps of FIG. 4E show the effect of such stimulations, visualizing the average neural firing recorded by each microelectrode. We find that within our cultures, firing rates of neurons surrounding the stimulation site are enhanced, with levels of activation proportional to the intensity of the stimulus.

We proceed by pairing optical stimulation with a four-well chip design to illustrate a parallel environment for control and selective stimulation experiments. To this end, ESC-MNs are seeded into four independent clusters on the MEA of FIG. 4F. Spontaneous activity is first recorded and processed to compute network correlations within wells and across wells. We see that recordings within the same cluster show high correlation scores. We then selectively apply optical stimulation to one well (#3), obtaining a highly synchronized response compared to the other wells.

Finally, we consider long-term electrophysiology applications. Complementary to short-term recordings that reveal transient and fast neural responses, longer recordings are necessary for investigating prolonged and slow plastic behaviors, such as facilitation, habituation, and long-term potentiation. [77-79] However, as underscored above, performing long-term recordings inside a high-humidity environment challenges the recording system with accelerated electronic degradation. We reduce liability via a modular design for which most of the delicate electronics are kept outside the incubation chamber. This is one reason why the instant modularity of the system is advantageous. It should be further noticed that in cases of failure, financial losses are minimized given the system's relatively low cost. We then demonstrate the continuous monitoring of ESC-MNs cultures over 24 h. As illustrated in FIG. 4G, an overall consistent level of spontaneous neural activity is observed, showing no sign of either hardware or culture-wise degradation throughout the recording. We emphasize that the need of daily media refreshment is the main reason for not extending the recording beyond 24 h in this experiment. Indeed, the same recording device, including the on-board amplifiers, has been actively utilized in incubators on an almost daily basis in our lab for over six months without catastrophic deterioration. This durability demonstrates the device's potential, upon coupling with an automated media refreshment system, for weeks-or months-long recordings, particularly useful for future computing applications where training/learning may be extensive. We also note that prolonged recordings inevitably produce high volumes of data (in the TB range), rendering post-processing via standard PC workstations cumbersome and time-consuming. This motivates the extension of our software to include functionalities for streamlining large-scale post analysis through cloud storage and HPC. Results in FIG. 4G are obtained by deploying such functionalities on the supercomputing facility Frontera at the Texas Advanced Computing Center.

Higher Dimensional Neural Systems: While 2D cultures allow for initial characterization and understanding of a networked cellular system, their lack of 3D organization does not fully capitalize on neurons' potential for enhanced connectivity, compute density, or miniaturization, nor can they recapitulate physiological architectures. Here, we demonstrate our platform in a higher-dimensional context, by considering both ex vivo tissue and 3D engineered mimics.

Organotypic Brain Slices: Organotypic cultures are prepared from slices of rat cortex that are 450 μm thick. While these compress to about 100 μm during incubation, they nevertheless preserve an intrinsic connectivity structure, which is lacking in the 2D monolayer cultures described above. At the same time, brain slices do not possess a clear 3D shape like the tissue mimics discussed below. To highlight this distinction, we consider organotypic cultures to be of intermediate dimension (2.5D). Relative to 2D cultures, experimentation with higher dimensional systems poses additional challenges, such as ensuring that the tissue is broadly and firmly in contact with the microelectrodes, as well as provisions for continuous media, oxygen, or drugs replacement. To address these challenges, we augment our system through a 3D printed fluidic interface that is modularly integrated via accessory posts protruding from the top acrylic layer (FIG. 5A). The printed apparatus hosts a pair of L-shape fluid delivery cannulas, connected to a peristaltic pump for continuous media delivery and aspiration. This can be further extended to support a “plug” system whereby a biocompatible nylon filter mesh can be gradually lowered towards the tissue to apply a gentle pressure onto it, thus enhancing contact across the electrodes surface.

Using this particular accessory, we perform longitudinal recordings in mature organotypic cortex cultures. As previously discussed, this preparation is commonly employed because it preserves much of the intrinsic tissue structure found in vivo, like cortical layers and corresponding cell types. Further, it facilitates in-depth culture manipulation via localized drug delivery, optical control, and cell identity imaging, which are difficult to perform in vivo. Cortical tissue is dissected from rat pups on post-natal day 5. These slice cultures are incubated for 26 days before transferring the slices to the MEA-chips for recording (protocol below). For this application, we utilize the curved MEA to conform to the natural morphology of the region of interest (FIG. 5B). Neural activity recorded from a 5 μm-MEA sample is presented in FIG. 5c, illustrating spontaneous spiking events as a raster plot, together with corresponding waveforms (spike cutouts). We use our software to isolate both multi-unit activity (MUA) and single-unit activity (SUA) across the electrode array, and find that MUA is highly synchronized across the cortical culture, consistent with significantly correlated bursting activity.

In addition to spontaneous recordings, we employ the same biphasic stimulation protocol as described above to study the tissue neural response. Activity in the sample is found to be strongly driven by the electrical stimulation (the red dashed line overlaid to the raster plot) across the entire 128-channel array. We note here that the use of curved MEAs also allows for consistent alignment of electrode sites to anatomical points of reference across the tissue. This enhances yield and increases usability, making it easier to reliably place and align the tissue to enhance experimental consistency across samples. Good alignment and yield are demonstrated in FIG. 5B, where recorded activity (firing rates) is mapped to the electrodes' physical locations.

Engineered Tissue Mimics: While organotypic slices are representative of the intrinsic connectivity of brain tissue, engineered mimics potentially allow the realization of 3D neural architectures of desired size, topology, and composition. This, combined with custom MEAs for interfacing, provides a unique opportunity to extend applications from neuroscience to engineering devices for sensing, processing, and computing. We present here two different methods of bio-fabricating 3D engineered neural tissue mimics (NTMs), and demonstrate their integration in our platforms.

We first extend our 2D monolayer cultures to 3D NTMs by mixing ESC-MNs with 6 mg mL-1 Matrigel (extracellular matrix, ECM). Here, we showcase the control over our NTM geometry by seeding the cell-ECM mixture into PDMS (polydimethylsiloxane) molds placed on our multi-well MEAs (FIG. 3C). Molds are fabricated with cavities of different shapes, allowing the mixtures to polymerize into 3D neural constructs of the prescribed configuration (FIG. 5E). To confirm the success of the NTM fabrication, fluorescence imaging is performed to visualize live neurons' GFP signals. As illustrated in FIG. 5E, neurons are evenly distributed within the constructs, and network formation is observed. We then deploy our platform for electrophysiology recording. FIG. 5F illustrates the activity obtained from an NTM sample, where spikes and synchronized events are detected across 17 out of 128 channels (a channel is defined as active when detected neural spikes exceed 0.5 Hz). We note that this percentage (˜15%) is lower than observed for 2D neural cultures (>80%, FIG. 4D) or 2.5D organotypic brain slices (>50%, FIG. 5C). This is because in NTM samples, neurons at relatively low density are distributed in 3D space so that the number of cells residing at the bottom of the tissue is limited, rendering activity less detectable by the electrodes.

Alternatively, NTMs can also be realized through the creation of 3D neural spheroids. Formed from embryonic stem cells and further differentiated into neural lineage, these spheroids recapitulate physiologically relevant features such as cellular and ECM composition. [80] We fabricate the neural spheroids by first obtaining embryoid bodies following the standard SFEBq method,[81] and then differentiating them into cortical lineage by selective Shh (via cyclopamine) and Wnt pathway (via IWP-2) inhibition. The resulting spheroids are maintained in a suspension culture for 20 days before seeding on MEAs for recording (FIG. 5G). Here, in order to showcase the native platform compatibility with commercial MEAs, we specifically employ one from the Multichannel system 60MEA series. FIG. 5G depicts a microscopic image taken on day 7 after seeding, illustrating a spheroid extending neurites on the commercial MEA. Corresponding recordings on day 12 after seeding demonstrate the successful detection of neural firing (FIG. 5H).

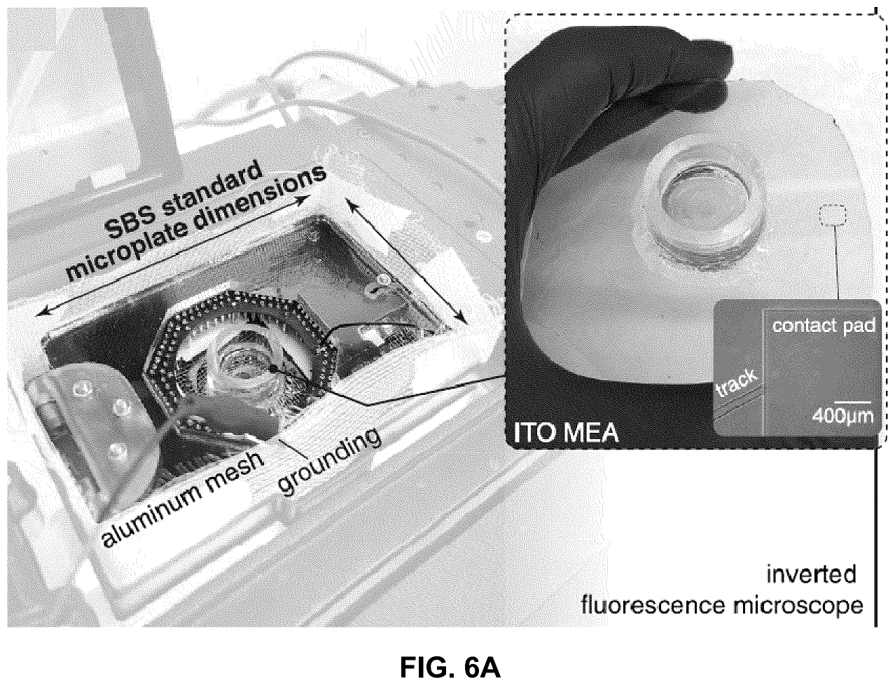

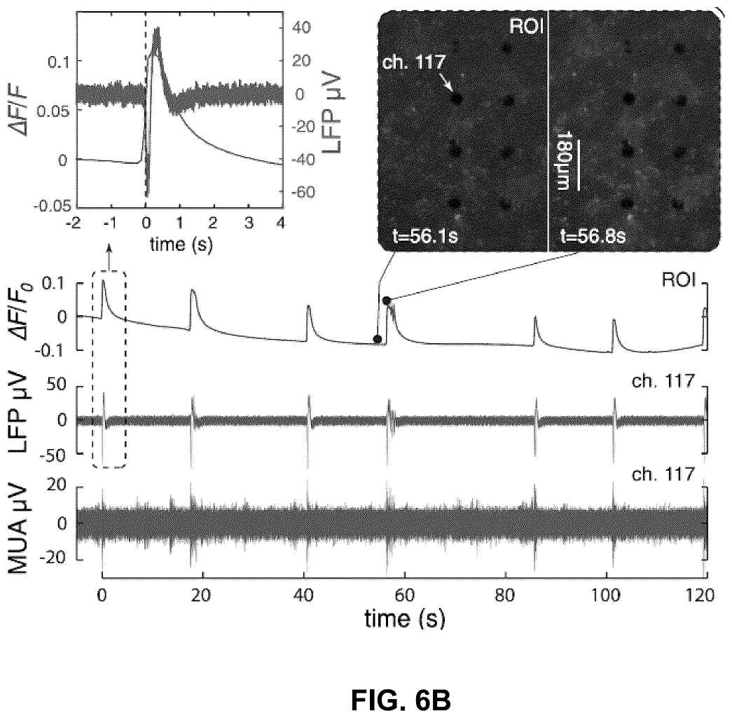

Concurrent Calcium Imaging: A unique aspect of a fully-customizable system is that its components can be continuously adapted to comply with existing industrial standards or new standards as they emerge while retaining the same core infrastructure. We showcase this here by reshaping our platform to allow concurrent calcium imaging. We alter the design of FIG. 1A to match the dimensions of a standard 96-well microplate (Society for Biomolecular Screening, SBS), enabling its integration with inverted microscopic chambers. As seen in FIG. 6A, a new PCB interface that accommodates a single 128-channel recording headstage (rather than two 64-channel headstages as in FIG. 1B) is mounted onto reshaped acrylic layers to deliver a compact, SBS-compatible layout.

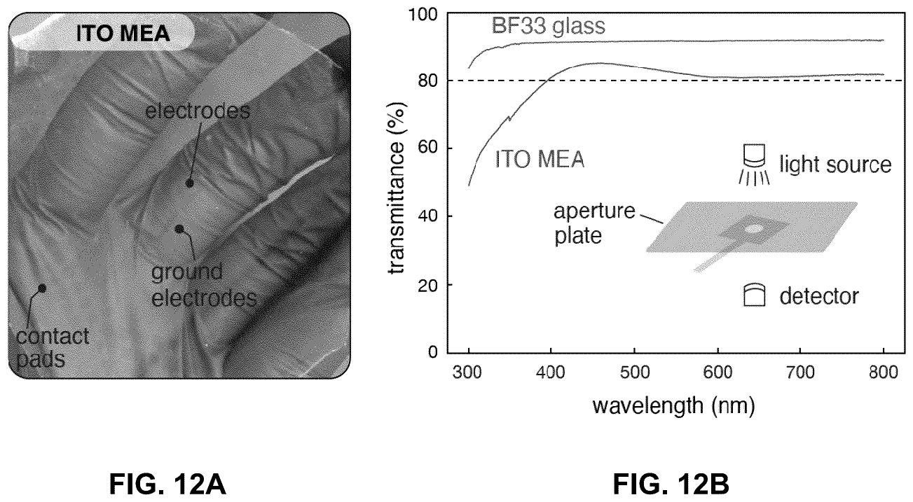

Upon integration within the microscope chamber (FIG. 6A), we proceed with testing the simultaneous electrophysiology and calcium imaging in 2D cultures of primary hippocampal neurons (PNs). PN cells utilized in this experiment are collected from embryonic rat brains (E18-E19) and cultured on MEAs following the same ESC-MNs seeding protocol. PNs are chosen because, unlike ESC-MNs, they do not express constitutive fluorescent reporters, and therefore allow the undisturbed visualization of the calcium signal. For this specific application, we employ transparent MEAs. These are microfabricated following the same procedure described herein, except for the addition of an annealing process after the ITO deposition to enhance transmittance and conductivity. [82] Annealing is carried out by placing the MEA sample in a vacuum chamber at 450° C. for 1 h, leading to ITO transmittance of ˜80% and resistivity of ˜4.5× 10-4 Ωcm (FIGS. 12A-12B), enabling both optical electrical measurements.

We perform concurrent measurements 12 days after seeding. Cells are loaded with the calcium indicator Oregon Green 488, following the manufacturer's recommendations (described below). The loaded sample is then placed in our platform within the microscopic chamber, ready for measurement. After setting the laser source, video, and electrophysiology recordings of the culture's spontaneous activity are simultaneously acquired, and offline analyzed. A representative example of post-processed signals is illustrated in FIG. 6B. Calcium activity is characterized through changes in fluorescent intensity (ΔF/F0, defined below) within a region of interest (ROI), which in turn determines the microelectrodes to be considered. A relatively large ROI is selected, thus considering collective network behavior rather than single-or few-neuron dynamics. Each peak in the fluorescence plot corresponds to a culture-wide burst event, during which we can observe a higher-intensity calcium signal across the network, relative to the rest state (illustrated in the inset images). Detected electrical signals are processed separately to reveal local field potentials (LFPs) and MUA during bursts, demonstrating that recording is not affected by laser-induced artifacts. Further, LFPs, MUA, and calcium signal spikes are found to precisely align temporally, confirming simultaneous neural responses across modalities.

Calcium imaging of millimeter-sized neural networks has been shown effective in revealing spatial connectivity and activity patterns in various physiological and topographical conditions.[83] Complemented by electrophysiology, the concurrent measurement setup provides the opportunity to advance such analyses via multi-modal, high spatiotemporal resolution information. Further, this approach is not necessarily limited to population-level imaging, but single-cell concurrent measurements may be incorporated for a detailed understanding of each cell's role in neural subnetworks. This research avenue will require higher density electrodes and a finer resolution imaging apparatus, and will be the subject of future investigation.

Portability, Robustness, and Reproducibility: Finally, we expand our discussion to emphasize our platform's portability, robustness, and reproducibility, key factors for broad dissemination.

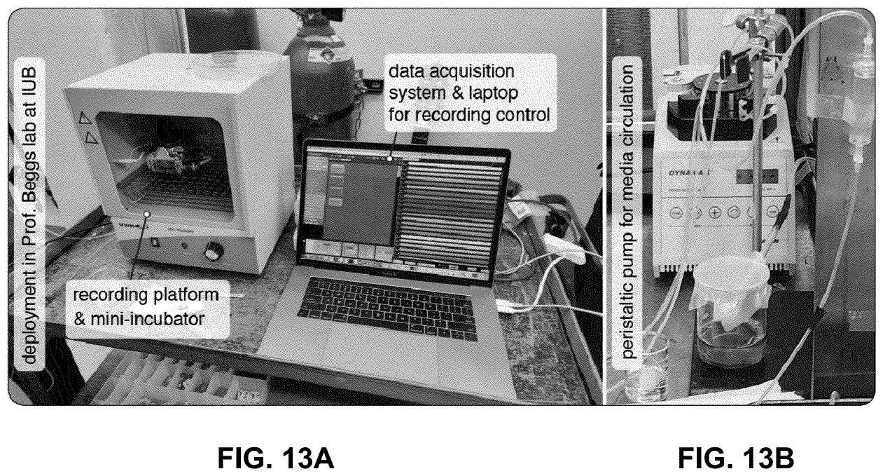

Electrophysiology measurements are delicate and sensitive to hardware setup and testing environments, rendering the portability of recording solutions rarely reported or discussed (FIG. 2). Here, we demonstrate this capability through off-site recordings of organotypic brain slices. To this end, a platform in use is disassembled and ground transported elsewhere (˜180 miles, ˜3 h drive). As illustrated in FIGS. 13A-13B, the disclosed system, after transportation, is readily reassembled and deployed for experiments within half an hour. This demonstration compounds the above-described case studies, further underscoring the robustness, versatility, and accuracy of the disclosed systems across a variety of conditions.

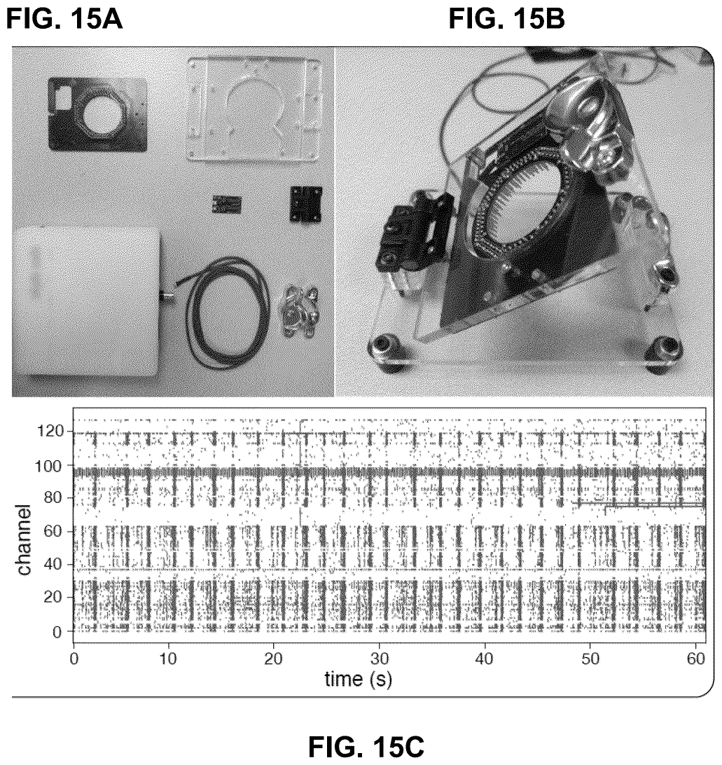

These favorable features led us to collect nearly 1000 h of recording experiments in 1 year, amounting to tens of TB of data. This ability has motivated others to adopt our systems as an alternative to commercial devices, providing testing opportunity of the independent reproducibility and implementation of the platforms. As but one example, see FIGS. 15A-15C.

This example demonstrates a versatile, scalable, and multi-modal electrophysiology solution, delivering a customizable signal pipeline stretching from in vitro neural substrates to cloud computing. Our approach significantly lowers barriers to entry through the open-sourcing of designs, software, and protocols, and via (over) tenfold cost reductions. The utility of our platforms is showcased through a comprehensive set of demonstrations involving a variety of cell types and stimulation modalities, 2D and 3D systems, concurrent imaging and long-term recording. This example provides a fundamental platform to broaden the electrophysiology domain, both in terms of users and applications, and motivates further innovation across scale and functionalities, paving the way for new biophysical discovery and in vitro technologies.

### Experimental Section

MEA Fabrication and Pt Deposition: The MEAs are fabricated on a set of borosilicate glass wafers (BOROFLOAT33) ranging from 3 to 6 in. (76.2-152.4 mm) in diameter. The process starts with the spin-coating of AZ5214E photoresist (PR) using a Headway PWM32 spin coater at a steady speed of 3000 rpm. Photolithography is then performed using the Heidelberg MLA 150 Maskless Aligner through a 375 nm laser source with a dose of 210 mJ cm-2. Developing is subsequently performed for 30 s using 1:4 diluted AZ400K developer, followed by a 50 s post-bake at 120° C. Next, the AJA sputter coater series is leveraged for constitutive material deposition. Ti and Pt are both deposited at 3 mT pressure, with respective power of 200 and 50 W, while ITO is deposited at 5 mT pressure, 80 W power, and under the airflow of argon and 3% oxygen. Samples are then submerged in acetone for lift-off. Oxford PECVD is then employed to apply a 500 nm passivation layer of Si₃N₄, which is produced with a supply of 20 sccm of SiH₄ and NH₃, respectively, under 650 mT pressure and 20 W power. The second photolithography process is carried out using the same aforementioned parameter settings. The exposed passivation material is removed using Oxford Freon RIE with 30 sccm CF₄ as the etching gas. Finally, the sample is cleaned with acetone and diced using a wafer cutter. An illustration of the fabrication process is found in FIG. 8.

To electroplate the Pt black, 100 mL of chloroplatinic acid that contained 1 g of hydrogen hexachloroplatinic hydrate, 0.01 g of lead (II) acetate trihydrate, and 0.25 mL of hydrochloric acid (Sigma-Aldrich) is first prepared and mixed.[69] 1 mL of mixed solution is added to each MEA and then electroplated, utilizing a Keithley 6221 current source to supply a DC current of density 4 nA μm-2, with the ground electrode being the anode and all other electrodes being the cathode.

Embryonic Stem Cell-Derived Motor Neuron Preparation: For the preparation of ESC-MNs, the optogenetic mouse ESC cell line ChR2^{H134R}-HBG3 Hb9-GFP [84] is differentiated following an established protocol. Briefly, mESCs are first cultured on a feeder layer of CF-1 mouse embryonic fibroblasts (Gibco), then the mESCs are suspended in an induction medium (advanced Dulbecco's Modified Eagle Medium/Nutrient Mixture F-12 and Neurobasal media with 10% KnockOut serum replacement, 1% l-glutamine and 1% penicillin-streptomycin) in a low-adhesion cell culture dish for 2 days to allow for the spontaneous formation of embryoid bodies (EBs). The EBs are then suspended in a differentiation medium (induction medium supplemented with 2% B-27, 1% N-2, 1% insulin transferrin selenium, 1 μm retinoic acid, and 1 μm smoothened agonist) for up to 5 days. GFP expression is monitored daily to confirm the differentiation into motor neurons. After differentiation, the EBs are dissociated using Accutase and the resulting single-cell suspension is seeded on the MEAs at a density of 5000 mm-2, following the seeding protocol detailed below. The neurons are kept in maintenance media (Neurobasal plus media with 2% B-27 plus, 1% 1-glutamine, 1% penicillin-streptomycin). In the first 4 days after seeding, maintenance medium is supplemented with growth factors (BDNF, GDNF, CNTF, NT-3, Forskolin, and IBMX) to promote neurite outgrowth and cell viability. [86]

Primary Neuron Preparation: Primary hippocampal neurons are dissected from time-pregnant rats at E18-E19, and put in MilliQ water (pH 7.4, 4° C.) supplemented with 1.16% Na₂SO₄, 0.52% K₂SO₄, 0.24% 4-(2-hydroxyethyl)-1-piperazineethanesulfonic acid (HEPES), 0.18% d-glucose, and 0.1% MgCl₂ 6H₂O. Hippocampal neurons are further dissociated and kept in Minimum Essential Medium (MEM) Eagle's with Earle's Balanced Salt Solution (BSS) without l-glutamine, supplemented with glucose, 100 mm sodium pyruvate, 200 mm l-glutamine, and 100 U mL-1 penicillin and streptomycin. Seeding and subsequent culture maintenance are similarly performed as for ESC-MNs.

Seeding on MEAs: To prepare the MEAs for neuron seeding, each of them is bonded with a small PDMS well around the electrode area (see also FIGS. 10A-10B for more details) through a biocompatible double-sided tape (Grace-Bio Labs, 620001). This well defined the neuron seeding area, and could be removed without damaging the MEA surface by applying an acetone wash. For culturing dissociated cultures (both ESC-MNs and PNs), MEAs are first surface-treated using oxygen plasma, followed by an overnight coating of 0.1 mg mL-1 poly-d-Lysin (PDL, Gibco) at room temperature. After the PDL solution is removed, the MEAs are washed with phosphate buffered saline (PBS) and allowed to air dry completely. MEAs re then incubated with 20 μg mL-1 laminin solution (Sigma Aldrich) at room temperature overnight, and the coating solution is removed immediately before cell seeding without air-drying.

Organotypic Brain Slice Preparation: Following an established protocol, [87] brains from postnatal day five rats re sliced into 400 μm slices containing somatosensory cortex. Slices are then incubated in a humidified atmosphere with 5% CO₂ at 37° C. in the culture medium, which consisted of 2:4 MEM, 1:4 Hank's BSS, 1:4 Horse serum, 4 mm glutamine, and 1:100 penicillin/streptomycin. Half of the medium is replaced every 3 days. Two weeks after slice preparation, slices are removed from the incubator and placed on the curved MEA for recording. During recording, oxygenated culture medium is pumped into and out of the MEA well via a peristaltic pump (FIGS. 13A-13B). An inline heater is placed immediately before the well to warm the medium to 37° C. Both prior to and after each experiment, distilled water and isopropyl alcohol are run through the entire fluid line to clean them of any biological debris.

Loading Calcium Indicator: Calcium imaging is performed by first loading the neuronal culture with a cell-permeable fluorescent calcium indicator, Oregon Green 488 BAPTA-1 (AAT Bioquest). To do this, the calcium indicator is diluted in the neural maintenance medium at a final concentration of 4 μm, and then supplied to the culture to replace the old medium. Subsequently, the culture is incubated for 40 min in a humidified environment with 5% CO₂ at 37° C. Right before the measurement, the culture is washed twice with PBS and supplied with fresh medium. Fluorescence Intensity Measurement: Spontaneous calcium activity is acquired by employing a fluorescent microscope (Nikon Eclipse Ti) and a fluorescein isothiocyanate (FITC) filter with an excitation/emission wavelength of 490/525 nm. Recording is conducted under a 488 nm laser illumination and at an original frame rate of 100 fps. The recorded video is then down-sampled to measure the fluorescence intensity using the open-source software ImageJ. The variation of the fluorescence intensity is defined through the normalized form as ΔF/F₀=(F-F₀)/F₀, where F and F₀ are the instantaneous and initial fluorescence intensity, respectively.

Signal Post-Processing: This subsection provides an overview of the signal post-processing procedure used in the open-source software. For each process, the corresponding modules (algorithms) are referenced to enhance the reproducibility of the procedure. The data recorded from the Open Ephys acquisition terminal is first loaded into the analysis pipeline using module: DataManager and DataLoader, which embed the native Python interface provided by Open Ephys for binary data conversion. The raw signal is then filtered using a third-order bandpass Butterworth filter with cutting frequencies at 200 and 3000 Hz (module: ButterBandpass). Spike detection is subsequently performed by applying a threshold at 5median {|x|}/0.6745, where x denotes the bandpass-filtered signal (module: ThresholdCutoff). Spike sorting is performed using the PCA decomposition method provided in the module: SpikeSorting. The detected spike train is also used to obtain statistical information for each recording channel, such as the firing rate (module: firing_rates) and the signal-to-noise ratio (module: spike_amplitude_to_background_noise). Further, the correlation coefficients across multiple channels are calculated by importing external packages from Elephant [88] (module: elephant.spike_train_correlation.correlation_coefficient), with a bin size of 10 ms.

### References Corresponding to Example 1

X. Zhang et al. “Mind In Vitro Platforms: Versatile, Scalable, Robust, and
Open Solutions to Interfacing with Living Neurons.” Adv. Sci. 2023, U.S. Pat. No. 2,306,826 (Dec. 31, 2023).
[1] R. R. Llinás, Science 1988, 242, 1654.
[2] E. Salinas, T. J. Sejnowski, Nat. Rev. Neurosci. 2001, 2, 539.
[3] K. C. Kanning, A. Kaplan, C. E. Henderson, Annu. Rev. Neurosci. 2010, 33, 409.
[4] A. L. Hodgkin, A. F. Huxley, Nature 1939, 144, 710.
[5] D. A. McCormick, B. W. Connors, J. W. Lighthall, D. A. Prince, J. Neurophysiol. 1985, 54, 782.
[6] E. R. Kandel, W. Spencer, F. Brinley Jr, J. Neurophysiol. 1961, 24, 225.
[7] M. Scanziani, M. Häusser, Nature 2009, 461, 930.
[8] B. Connors, M. Gutnick, D. Prince, J. Neurophysiol. 1982, 48, 1302.
[9] J. M. Beggs, D. Plenz, J. Neurosci. 2003, 23, 11167.
[10] C. Forro, D. Caron, G. N. Angotzi, V. Gallo, L. Berdondini, F. Santoro, G. Palazzolo, G. Panuccio, Micromachines 2021, 12, 124.
[11] H. Shin, S. Jeong, J.-H. Lee, W. Sun, N. Choi, I.-J. Cho, Nat. Commun. 2021, 12, 492.
[12] C.-F. V. Latchoumane, L. Jackson, M. S. E. Sendi, K. F. Tehrani, L. J. Mortensen, S. L. Stice, M. Ghovanloo, L. Karumbaiah, Sci. Rep. 2018, 8, 10957.
[13] G. J. Pagan-Diaz, K. P. Ramos-Cruz, R. Sam, M. E. Kandel, O. Aydin, M. T. A. Saif, G. Popescu, R. Bashir, Proc. Natl. Acad. Sci. U.S.A 2019, 116, 25932.
[14] K. Nahrstedt, N. Shanbhag, V. Adve, N. Amato, R. R. Choudhury, C. Gunter, N. S. Kim, O. Milenkovic, S. Mitra, L. Varshney, Y. Vlasov, S. Adve, R. Bashir, A. Cangellaris, J. DiCarlo, K. Driggs-Campbell, N. Feamster, M. Gazzola, K. Karahalios, S. Koyejo, P. Kwiat, B. Li, N. Mehr, R. Mehra, A. Miller, D. Rus, A. Schwing, A. Shrivastava, arXiv: 2210.08974, 2022.
[15] J. W. Park, B. Vahidi, A. M. Taylor, S. W. Rhee, N. L. Jeon, Nat. Protoc. 2006, 1,2128.
[16] L. Pan, S. Alagapan, E. Franca, G. J. Brewer, B. C. Wheeler, J. Neural Eng. 2011, 8, 046031.
[17] A. Gladkov, Y. Pigareva, D. Kutyina, V. Kolpakov, A. Bukatin, I. Mukhina, V. Kazantsev, A. Pimashkin, Sci. Rep. 2017, 7, 15625.
[18] S. Joo, J. Lim, Y. Nam, BioChip J. 2018, 12, 193.
[19] L. Smirnova, B. S. Caffo, D. H. Gracias, Q. Huang, I. E. Morales Pantoja, B. Tang, D. J. Zack, C. A. Berlinicke, J. L. Boyd, T. D. Harris, E. C. Johnson, B. J. Kagan, J. Kahn, A. R. Muotri, B. L. Paulhamus, J. C. Schwamborn, J. Plotkin, A. S. Szalay, J. T. Vogelstein, P. F. Worley, T. Hartung, Front. Sci. 2023, 1, 1017235.
[20] J. Soriano, Biophysica 2023, 3, 181.
[21] Y. Nam, B. C. Wheeler, Crit. Rev. Bioeng. 2011, 39, 45.
[22] A. Stett, U. Egert, E. Guenther, F. Hofmann, T. Meyer, W. Nisch, H. Haemmerle, Anal. Bioanal. Chem. 2003, 377, 486.
[23] E. R. McConnell, M. A. McClain, J. Ross, W. R. LeFew, T. J. Shafer, Neurotoxicology 2012, 33, 1048.
[24] M. Eichler, H.-G. Jahnke, D. Krinke, A. Müller, S. Schmidt, R. Azendorf, A. A. Robitzki, Biosens. Bioelectron. 2015, 67, 582.
[25] J. Müller, M. Ballini, P. Livi, Y. Chen, M. Radivojevic, A. Shadmani, V. Viswam, I. L. Jones, M. Fiscella, R. Diggelmann, A. Stettler, U. Frey, D. J. Bakkuma, A. Hierlemann, Lab Chip 2015, 15, 2767.
[26] Harvard Bioscience, Inc., https://www.multichannelsystems.com (accessed: December 2023).
[27] Axion Biosystems, https://www.axionbiosystems.com (accessed: December 2023).
[28] A. P. Buccino, M. E. Lepperød, S.-A. Dragly, P. Häfliger, M. Fyhn, T. Hafting, J. Neural Eng. 2018, 15, 055002.
[29] G. M. O'Leary, I. Khramtsov, R. Ramesh, A. Perez-Ignacio, P. Shah, H. M. Chameh, A. Gierlach, R. Genov, T. Valiante, bioRxiv 2022.
[30] D. Kim, H. Kang, Y. Nam, Lab Chip 2020, 20, 3410.
[31] S. Middya, V. F. Curto, A. Fernández-Villegas, M. Robbins, J. Gurke, E. J. Moonen, G. S. Kaminski Schierle, G. G. Malliaras, Adv. Sci. 2021, 8, 2004434.
[32] I. A. Weaver, A. W. Li, B. C. Shields, M. R. Tadross, J. Neural Eng. 2022, 19, 024001.
[33] C. Black, J. Voigts, U. Agrawal, M. Ladow, J. Santoyo, C. Moore, S. Jones, J. Neural Eng. 2017, 14, 035002.
[34] Intan Technologies, https://intantech.com (accessed: December 2023).
[35] D. Kuzum, H. Takano, E. Shim, J. C. Reed, H. Juul, A. G. Richardson, J. De Vries, H. Bink, M. A. Dichter, T. H. Lucas, D. A. Coulter, E. Cubukcu, B. Litt, Nat. Commun. 2014, 5, 5259.
[36] A. Scott, K. Weir, C. Easton, W. Huynh, W. J. Moody, A. Folch, Lab Chip 2013, 13,527.
[37] E. Muller, A. P. Davison, T. Brizzi, D. Bruederle, M. J. Eppler, J. Kremkow, D. Pecevski, L. Perrinet, M. Schmuker, P. Yger, Front. Neuroinform. Conference Abstract: Neuroinformatics 2009, https://doi.org/10.3389/conf.neuro.11.2009.08.104.
[38] F. Pedregosa, G. Varoquaux, A. Gramfort, V. Michel, B. Thirion, O. Grisel, M. Blondel, P. Prettenhofer, R. Weiss, V. Dubourg, J. Vanderplas, A. Passos, D. Cournapeau, M. Brucher, M. Perrot, É. Duchesnay, J. Mach. Learn. Res. 2011, 12, 2825.
[39] N. Cermak, M. A. Wilson, J. Schiller, J. P. Newman, bioRxiv 2019, 757716.
[40] A. Bansal, S. Shikha, Y. Zhang, Nat. Biomed. Eng. 2023, 7, 349.
[41] Med64, https://www.med64.com/products/advantages/(accessed: December 2023).
[42] M. Cantarelli, B. Marin, A. Quintana, M. Earnshaw, R. Court, P. Gleeson, S. Dura-Bernal, R. A. Silver, G. Idili, Philos. Trans. R. Soc., B 2018, 373, 1.
[43] K. Voitiuk, J. Geng, M. G. Keefe, D. F. Parks, S. E. Sanso, N. Hawthorne, D. B. Freeman, R. Currie, M. A. Mostajo-Radji, A. A. Pollen, T. J. Nowakowski, S. R. Salama, M. Teodorescu, D. Haussler, J. Neural Eng. 2021, 18, 34666315.
[44] L. Paninski, J. P. Cunningham, Curr. Opin. Neurobiol. 2018, 50, 232.
[45] J. L. Rossello, V. Canals, A. Oliver, M. Alomar, A. Morro, in Proceedings of the 2014 29th Conference on Design of Circuits and Integrated Systems, DCIS, IEEE, Piscataway, NJ 2014.
[46] R. Quian Quiroga, S. Panzeri, Nat. Rev. Neurosci. 2009, 10, 173.
[47] J. Thomas, J. Jin, J. Dauwels, S. S. Cash, M. B. Westover, IEEE Inter-national Conference on Acoustic Speech Signal Processing, IEEE, Piscataway, NJ 2018, pp. 970-974.
[48] B. Depasquale, D. Sussillo, L. F. Abbott, M. M. Churchland, Neuron 2023, 111, 631.
[49] J. M. Beggs, N. Timme, Front. Physiol. 2012, 3, 163.
[50] K. Heiney, O. Huse Ramstad, V. Fiskum, N. Christiansen, A. Sandvig, S. Nichele, I. Sandvig, Front. Comput. Neurosci. 2021, 15, 611183.
[51] S. Benjaminsson, D. Silverstein, P. Herman, P. Melis, V. Slavni, M. Spasojevićc, A. Lansner, Technical Report. Brussels: Partnership for Advanced Computing in Europe (PRACE), 2012, https://urn.kb.se/resolve?urn=urn: nbn: se: kth: diva-67472.
[52] S. Dura-Bernal, B. A. Suter, P. Gleeson, M. Cantarelli, A. Quintana, F. Rodriguez, D. J. Kedziora, G. L. Chadderdon, C. C. Kerr, S. A. Neymotin, R. A. McDougal, M. Hines, G. M. Shepherd, W. W. Lytton, eLife 2019, 8, e44494.
[53] Y. Babuji, A. Woodard, Z. Li, D. S. Katz, B. Clifford, R. Kumar, L. Lacinski, R. Chard, J. Wozniak, I. Foster, M. Wilde, K. Chard, in 28th ACM Int. Symp. on High-Performance Parallel and Distributed Computing (HPDC), Association of Computing Machinery, New York, NY 2019, pp. 1-8. https://doi.org/10.1145/3307681.3325400.
[54] A. P. Buccino, O. Winter, D. Bryant, D. Feng, K. Svoboda, J. H. Siegle, J. Neural Eng. 2023, 20, 056009.
[55] G. Hilgen, M. Sorbaro, S. Pirmoradian, J. O. Muthmann, I. E. Kepiro, S. Ullo, C. J. Ramirez, A. Puente Encinas, A. Maccione, L. Berdondini, V. Murino, D. Sona, F. Cella Zanacchi, E. Sernagor, M. H. Hennig, Cell Rep. 2017, 18, 2521.
[56] P. Yger, G. L. Spampinato, E. Esposito, B. Lefebvre, S. Deny, C. Gardella, M. Stimberg, F. Jetter, G. Zeck, S. Picaud, J. Duebel, O. Marre, eLife 2018, 7, e34518.
[57] A. Collette, Python and HDF5: Unlocking Scientific Data, O'Reilly Media, Inc., Sebastopol, CA 2013.
[58] G. Arakelyan, G. Soghomonyan, The Aim Team, Aim, https://github. com/aimhubio/aim (accessed: December 2023).
[59] D. G. Moore, G. Valentini, S. I. Walker, M. Levin, in 2017 IEEE Symp. Series on Computational Intelligence (SSCI), IEEE, Piscataway, NJ 2017, pp. 1-8.
[60] P. Wollstadt, J. Lizier, R. Vicente, C. Finn, M. Martinez-Zarzuela, P. Mediano, L. Novelli, M. Wibral, J. Open Source Software 2019, 4, 1081.
[61] S. Garcia, D. Guarino, F. Jaillet, T. Jennings, R. Pröpper, P. L. Rautenberg, C. C. Rodgers, A. Sobolev, T. Wachtler, P. Yger, A. P. Davison, Front. Neuroinf. 2014, 8, 10.
[62] M. Stimberg, R. Brette, D. F. Goodman, eLife 2019, 8, e47314.
[63] N. A. Steinmetz, C. Aydin, A. Lebedeva, M. Okun, M. Pachitariu, M. Bauza, M. Beau, J. Bhagat, C. Böhm, M. Broux, S. Chen, J. Colonell, R. J. Gardner, B. Karsh, F. Kloosterman, D. Kostadinov, C. Mora-Lopez, J. O'Callaghan, J. Park, J. Putzeys, B. Sauerbrei, R. J. J. van Daal, A. Z. Vollan, S. Wang, M. Welkenhuysen, Z. Ye, J. T. Dudman, B. Dutta, A. W. Hantman, K. D. Harris, et al., Science 2021, 372, eabf4588.
[64] M. Pachitariu, S. Sridhar, C. Stringer, bioRxiv 2023, 2023.
[65] L. Berdondini, K. Imfeld, A. Maccione, M. Tedesco, S. Neukom, M. Koudelka-Hep, S. Martinoia, Lab Chip 2009, 9, 2644.
[66] E. S. Lein, M. J. Hawrylycz, N. Ao, M. Ayres, A. Bensinger, A. Bernard, A. F. Boe, M. S. Boguski, K. S. Brockway, E. J. Byrnes, L. Chen, L. Chen, T.-M. Chen, M. C. Chin, J. Chong, B. E. Crook, A. Czaplinska, C. N. Dang, S. Datta, N. R. Dee, A. L. Desaki, T. Desta, E. Diep, T. A. Dolbeare, M. J. Donelan, H.-W. Dong, J. G. Dougherty, B. J. Duncan, A. J. Ebbert, G. Eichele, et al., Nature 2007, 445, 168.
[67] S. Ito, F.-C. Yeh, E. Hiolski, P. Rydygier, D. E. Gunning, P. Hottowy, N. Timme, A. M. Litke, J. M. Beggs, PloS One 2014, 9, e105324.
[68] A. Levina, V. Priesemann, Nat. Commun. 2017, 8, 15140.
[69] K. Mathieson, S. Kachiguine, C. Adams, W. Cunningham, D. Gunning, V. O'shea, K. Smith, E. Chichilnisky, A. Litke, A. Sher, M.M. Rahman, IEEE Trans. Nucl. Sci. 2004, 51, 2027.
[70] G. J. Pagan-Diaz, J. Drnevich, K. P. Ramos-Cruz, R. Sam, P. Sengupta, R. Bashir, Sci. Rep. 2020, 10, 12460.
[71] Multichannel Systems Microelectrode Array User Manual, https://www.multichannelsystems.com/downloads/documentation (accessed: December 2023).
[72] E. Estévez-Priego, M. Moreno-Fina, E. Monni, Z. Kokaia, J. Soriano, D. Tornero, Stem Cell Rep. 2023, 18, 205.
[73] E. S. Boyden, F. Zhang, E. Bamberg, G. Nagel, K. Deisseroth, Nat. Neurosci. 2005, 8, 1263.
[74] D. A. Wagenaar, J. Pine, S. M. Potter, J. Neurosci. Methods 2004, 138, 27.
[75] M. Toivanen, A. Pelkonen, M. Mäkinen, L. Ylä-Outinen, L. Sukki, P. Kallio, M. Ristola, S. Narkilahti, Front. Neurosci. 2017, 11, 606.
[76] S. R. Kelso, A. H. Ganong, T. H. Brown, Proc. Natl. Acad. Sci. U.S.A 1986, 83,5326.
[77] M. Niedringhaus, X. Chen, R. Dzakpasu, PloS One 2015, 10, e0129324.
[78] Y. Jiang, A. M. VanDongen, eNeuro 2021, 8, 6.
[79] L. Hermans, M. Kaynak, J. Braun, V. L. Ríos, C.-L. Chen, A. Friedberg, S. Günel, F. Aymanns, M. S. Sakar, P. Ramdya, Nat. Commun. 2022, 13, 5006.
[80] P. Zhuang, A. X. Sun, J. An, C. K. Chua, S. Y. Chew, Biomaterials 2018,154, 113.
[81] M. Eiraku, K. Watanabe, M. Matsuo-Takasaki, M. Kawada, S. Yonemura, M. Matsumura, T. Wataya, A. Nishiyama, K. Muguruma, Y. Sasai, Cell Stem Cell 2008, 3, 519.
[82] Y. Hu, X. Diao, C. Wang, W. Hao, T. Wang, Vacuum 2004, 75, 183.
[83] M. Montalà-Flaquer, C. F. López-León, D. Tornero, A. M. Houben, T. Fardet, P. Monceau, S. Bottani, J. Soriano, iScience 2022, 25, 105680.
[84] S. G. Uzel, R. J. Platt, V. Subramanian, T. M. Pearl, C. J. Rowlands, V. Chan, L. A. Boyer, P. T. So, R. D. Kamm, Sci. Adv. 2016, 2, e1501429.
[85] C.-Y. Wu, D. Whye, R. W. Mason, W. Wang, J. Visualized Exp. 2012, 64, e3813.
[86] H. B. Peng, J.-F. Yang, Z. Dai, C. W. Lee, H. W. Hung, Z. H. Feng, C.-P. Ko, J. Neurosci. 2003, 23, 5050.
[87] A. Tang, D. Jackson, J. Hobbs, W. Chen, J. L. Smith, H. Patel, A. Prieto, D. Petrusca, M. I. Grivich, A. Sher, P. Hottowy, W. Dabrowski, A. M. Litke, J. M. Beggs, J. Neurosci. 2008, 28, 505.
[88] M. Denker, A. Yegenoglu, S. Grün, Neuroinformatics, 2018.

### Example 2: Details of Platform and Related Components

The hardware platform is modularly designed to support a range of recording configurations with 59, 128, 256 and 512 channels. While Table 1 showcases the aggregation of all components utilized in these configurations, only a subset of the list is necessary to assemble a specific prototype. As shown in Table 1, a series of Intan (RHD series) headstages are available with various recording capacities (32, 64, 128). In principle, users are free to select any combination of these headstages for each recording configuration. However, we note that different headstages may require different connectors and PCB footprints for interfacing with the MEA. In this Example 1-2, we employ three different connector series: Intan adapter board, Omnetics Nano Strip connector and Molex SlimStack connector, listed in descending order of cost. A trade-off is then found between cost and assembly difficulty: the more inexpensive options generally require finer surface mount soldering technique, which in turn results in additional manufacturing time. Therefore, to strike a balance between cost and manufacturing complexity, we present two different headstage and connector selections for each configuration in Table 2. Both options are natively compatible with our recording platform and similar recording qualities are observed during testing. Therefore, we provide PCB designs involving footprints for all three connector series, so that users can select based on their fabrication capability, and adopt these designs for their specific needs [3].

Finally, we include the list of components required for employing Intan bi-directional recording/stimulation controller in Table 3. As discussed in the Example 1, this controller is an alternative for downstream data collection if high resolution electrical stimulation is desired. We note a different series of headstage and SPI cable (RHS series) are needed here for bi-directional signal communication. Nevertheless, the RHS 32 headstage employs the same connector scheme as the RHD 32 counterpart, so that they are fully interchangeable in terms of interfacing with the PCB.

We characterize the impedance of our custom MEAs across a variety of electrode diameters. This characterization is performed on a specially designed 59-electrode MEA, where electrodes of four different sizes are arranged in separate quadrants. Microscopic image of this MEA is presented in FIG. 9, demonstrating electrode with diameters of 5, 10, 20 and 30 μm, respectively. The effect of Pt deposition on each electrode diameter is also demonstrated in FIG. 9.

In this section, we present more details about employing our MEAs for recording. In FIG. 10A, we illustrate the PDMS culture wells affixed on the MEA for seeding 2D neural cultures or 3D engineered tissues. The size and shape of these wells can be customized depending on the electrode arrangement, but they are generally larger than the area spanned by the electrode pattern. Finally, a glass ring is also glued on the MEA for holding culture media. In FIG. 10B, we demonstrate the use of 3D-printed stoppers for centering and holding MEAs of various sizes and shapes. This ensures a precise and reproducible loading and alignment of the chips, enhancing recording quality.

To demonstrate the recording capability and characterize the performance of our system, we present result and analysis from embryonic stem cell-derived motor neurons (ESC-MNs) in Example 1. To further showcase a consistent recording quality across different cell types, in this section, we present the same characterizations with primary neuron (PN) cultures.

With the utility of the rectangle and curved MEA layouts discussed in Example 1, we employ the perturbed layout of the 128-channel MEA (FIG. 3C) for culturing PN cells in this experiment. The spontaneous activity of the cultured sample (FIG. 11A) is visualized through the raster plot of FIG. 11B. Snapshots of the filtered signals are also plotted for representative channels, illustrating the detectability of both single unit activities as well as synchronized bursts events. We then quantify the recording quality by characterizing signal-to-noise ratio (SNR) across channels (FIG. 11C). Similar to the results from ESC-MNs (FIG. 4D), a SNR>5 is observed for almost all channels. Finally, we demonstrate the electrical stimulation of the culture by employing the same protocol described in FIG. 4E, where biphasic electric pulses are applied at either side of the culture for selective activation. The heat-maps in FIG. 11D showcase the average firing rate of each electrode, while providing visualizations of the spatial activity pattern in response to stimulation at different sites. Overall, through this experiment, consistent recording and stimulation characteristics are observed with PN cultures as in the case of ESC-MNs, demonstrating the stability and consistency of our system performance across different cell types.

The reconfigurability of the hardware system provides the opportunity of integrating electrophysiology with advanced microscopy. This is demonstrated in Example 1, where primary neurons are seeded on transparent, indium tin oxide (ITO) MEAs (FIG. 12A) for concurrent calcium imaging and electrophysiology recording. We fabricate the ITO MEA following the process presented in FIG. 8, and then characterize its transmittance using an ultraviolet-visible spectrophotometer (UV-Vis, Varian Cary5G). To this end, an aperture plate is mounted onto the ITO MEA, and then aligned so that one of the contact pads is fully covering the aperture. Results presented in FIG. 12B showcase that our ITO MEA consistently exhibits a transmittance of >80% for a range of visible spectrum wavelengths between 400 to 800 nm, making it suitable for fluorescence imaging of neurons [4].

The system is portable. To this end, essential equipment is transported, including the Open Ephys board, a 128-channel hardware platform, multiple MEAs, a mini-incubator and a laptop. These devices demand minimal lab-space and can be conveniently set up with a very small footprint (FIG. 13A). For long-term or organotypic tissue recordings where maintaining oxygen and pH balance are critical, our system is fully compatible with systems optimized for media exchange. This is achieved through connecting the fluidic interface of FIG. 5A to the peristaltic pump depicted in FIG. 13B. We note that this fluidic system is primarily designed for organotypic tissue experiments, in which samples are not preserved after one day of recording. In case of experiments involving cell cultures and much longer monitoring (weeks-long), the media refreshment system has to be carefully configured to ensure robustness, reliability, and a sterilized environment throughout the entire process.

Apart from portability, we further demonstrate that our system is readily reproducible. Leveraging our shared designs and protocols, a set of four different platforms built by different investigators is shown in FIG. 14A. Each one hosts a different PCB interface, demonstrated in recording systems with 59, 128, 256 and 512 channels, respectively. Moreover, with the capability of the 128 channel version extensively discussed in Example 1, here, we showcase the maximum recording capacity of our system. To this end, the 512-electrode MEA presented in FIG. 3B is seeded with ESC-MNs for testing. A 512 channel raster plot is presented in FIG. 14B, illustrating the spontaneous activity of the culture on day 7 after seeding. As can be seen from the plot, spikes and synchronized bursts are detected for almost all recording channels, demonstrating the stability and the functionality of our system at the maximum recording capacity.

Finally, we present an example of our system being reproduced by researchers with no previous experience. FIG. 15A-15B showcase a replica of our 128-channel recording solution. Following our open-protocol, this system is assembled with a total amount of 15 hours of work, and successfully validates with an ESC-MNs culture. Recordings carried out by this new system are presented in FIG. 15C.

### References for Example 2

[1] available at: open-ephys.github.io/acq-board-docs/.
[2] available at: intantech.com.
[3] Open-source Hardware available at: gazzolalab.github.io/MiV-OH/; Software: https://miv-os.readthedocs.io.
[4] S. Middya, V. F. Curto, A. Fernández-Villegas, M. Robbins, J. Gurke, E. J. Moonen, G. S. Kaminski Schierle, G. G. Malliaras, Advanced Science 2021, 8, 13 2004434.

### Example 3: Automated Systems

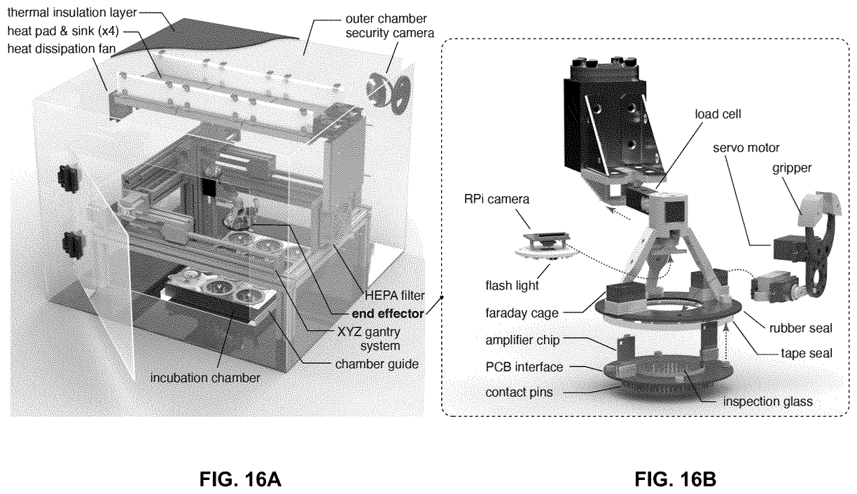

The system described in Examples 1-2 may be automated by incorporation of a robotic positioner that is programmable to electrically interface with the biological material via the MEA. Referring to FIGS. 16A-21, provided is an automated system for electrophysiologically interfacing with a biological material. The sterile platform may have a plurality of recess features to support a plurality of biological materials. Of course, within each recess feature, there may further comprise a plurality biological materials, such as by a configuration of MEA having a plurality of distinct regions to support a corresponding plurality of biological materials. For example, FIG. 3C illustrates an MEA configuration for four biological materials (via the multi-well configuration).

Electrical interconnects may connect the electrodes of the MEA with contact pads. The biological material is supported, at least in part, by the electrodes. Accordingly, during the culture, the biological material develops reliable electrical contact with at least one electrode. Each of the recess features (having biological material) may be covered by a removable lid. Robotic positioner has an end effector that can be multi-functional. For example, the PCB interface connected to the end effector is controllably positioned and aligned, via electronic instructions sent to the robotic positioner. Upon alignment, the PCB interface is brought into contact with contact pads, thereby establishing an electrical interface with the biological material (via electrodes, interconnects, contact pad). A downstream signal acquisition unit that is electronically connected to the biological material (via the PCT interface) can be used to store/transmit data and to control robotic positioner. As known in the art, a controller and processor are used together to control the robotic positioner, including via an electrophysiological interface scheme.

The robotic positioner also has a lid grip to interact with the lid(s). The end effector can further take input from one or more sensors, such as position sensor and/or force sensor, to controllably engage with lid to generate a lid-removed (where the underlying biological material is ready for interfacing) or a lid-sealed (where biological material is reliably sealed and ready for ongoing culture) configuration.

The system is highly modular and configurable. Biological materials, MEA and PCB are each and all readily tailored for an application of interest, such as to optimize spatial or temporal resolution, including by adjusting the number of electrode channels. In addition, the system may be configured to have a form-factor to fit within an incubation chamber (FIG. 16A). Various components are illustrated in FIG. 16B, including a lid grip and servo motor for robotic positioning of end effector. Also illustrated is optical interface, PCB interface and faraday cage to minimize unwanted electrical noise.

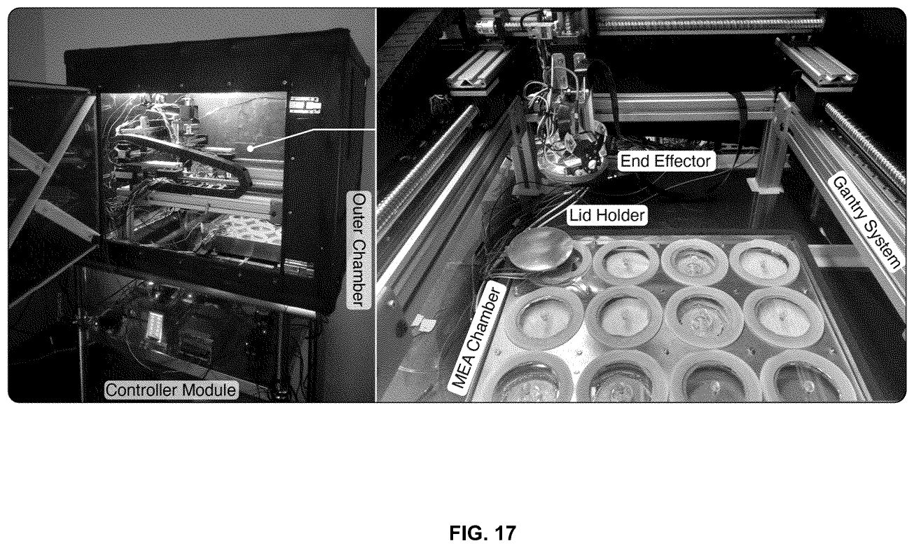

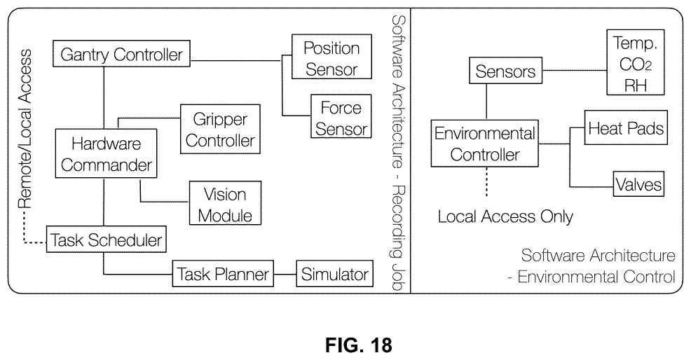

FIG. 17 are photographs of a system in an incubator ready for control by controller, with the right panel a close-up view of the end effector movable by robotic positioner via gantry system. FIG. 18 schematically illustrates software architecture implemented via controller and associated instructions of the electrophysiological interface scheme. The left side of the figure relates to task planning and automated handling of lid interaction and the right side for controlling any number of physiological parameters, such as T, CO₂ level, humidity, flow along with related sensors.

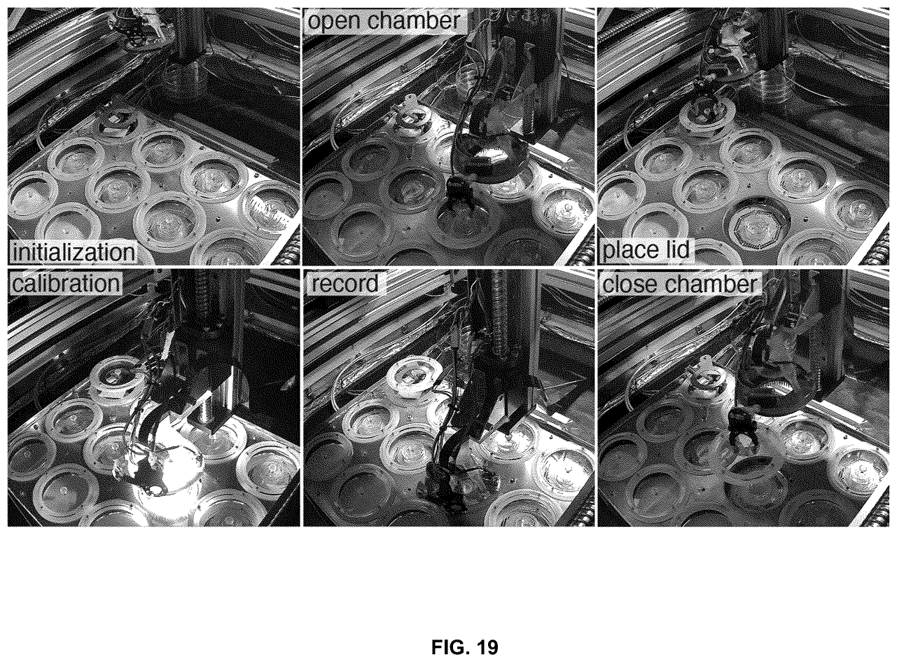

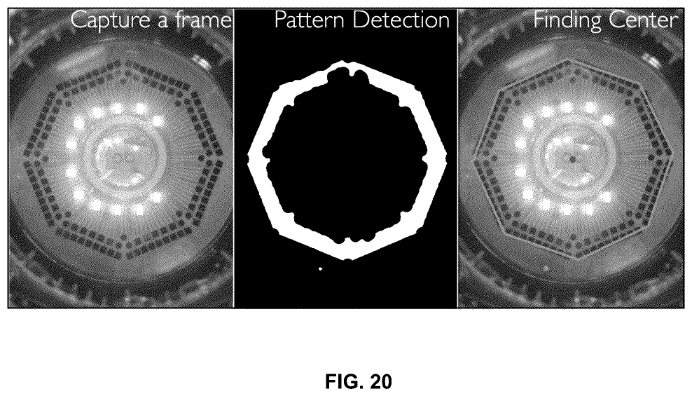

FIG. 19 summarizes the work flow process of the robotic positioning and electrical interfacing with biological tissue. FIG. 20 focuses on an automated optical alignment with optical detector that captures an image, including with optical markers (left panel), pattern detection (middle panel) and an algorithm to detecting a center point (right panel). Such reliable alignment is useful for both lid grip and electrical interfacing between PCB and MEA.

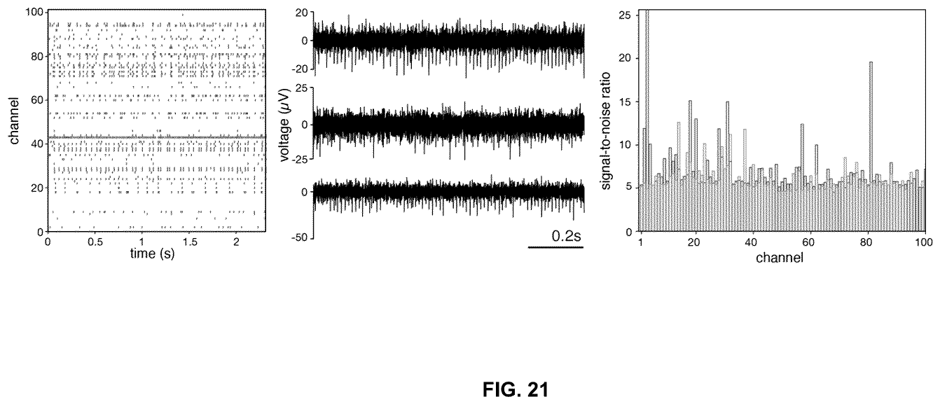

FIG. 21 provides experimental results of a system that is electrically interfaced with biological material, including for a high number of channels (left panel), electrical traces for various channels (middle panel), and good signal-to-noise ratio (SNR).

Also provide are methods of using any of the systems provided herein, including for a single manual-use system, and for fully automated systems having the robotic positioner.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure. Specific names of compounds are intended to be exemplary, as it is known that one of ordinary skill in the art can name the same compounds differently.

Every device, system, formulation, combination of components, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

TABLE 1
List of components used in constructing the recording
hardware along with their costs in USD.^{a}

| Component | Cost (USD) | Component | Cost (USD) |
|---|---|---|---|
| Open Ephys board | 3200 | Intan RHD 32ch Headstage | 940 |
| (self\|-assemble^{b}) | (~1000) | (self-assemble) | (~600) |
| Intan RHD 64ch Headstage | 1875 | Intan RHD 128ch Headstage | 2995 |
| (self-assemble) | (~800) | (self-assemble) | (~1200) |
| Omnetics connector^{c} | 85.21 | Molex connector^{d} | 1 |
| 36-pin adapter board | 255 | RHD SPI cable | 215 |
| RHD dual adaptor | 345 | POGO pins^{e} | ~0.6 |
| Other hardware^{f} | ~60 | | |

^{a}Online prices as of August 2023.
^{b}Self-assemble cost estimates only account for raw material costs, excluding external chip assembling service.
^{c}Omnectics 36 position dual row nano strip A79022-001.
^{d}Molex SlimStack 5024806410.
^{e}Mill-Max spring-loaded pogo pins with regular tail length 0914-2-15-20-77-14-11-0, with extended tail for electrical stimulation 0929-9-15-20-75-14-11-0.
^{f}Other hardware includes acrylic boards, hinge, latch, bumpers and PCB fabrication. We fabricate our PCBs externally through Oshpark LLC.

TABLE 2
Headstage, connector and cable selections to construct 59, 128, 256 and 512-channel recording
systems. Total cost of each configuration includes the costs of the components on the list
(headstage, connector and cable), an Open Ephys data acquisition board, the required amount
of pogo pins and other hardware accessories needed to construct the recording platform.

| | Headstage | Connector | Cable | Total cost | Total cost |
|---|---|---|---|---|---|
| Config. | options | options | options | (USD) | self-assembly (USD) |
| 59ch | 2 × RHD 32 | 2 × adapter board | 2 × SPI | ~6k | ~3.5k |
| | 1 × RHD 64 | 2 × Omnetics | 1 × SPI | ~5.5k | ~2.3k |
| 128ch | 2 × RHD 64 | 4 × Omnetics | 2 × SPI | ~7.7k | ~3.6k |
| | 1 × RHD 128 | 2 × Molex | 1 × SPI | ~6.5k | ~2.6k |
| 256ch | 4 × RHD 64 | 8 × Omnetics | 4 × SPI | ~12k | ~6k |
| | 2 × RHD 128 | 4 × Molex | 2 × SPI | ~9.7k | ~4.1k |
| 512ch | 8 × RHD 64 | 16 × Omnetics | 4 × (SPI + dual adapter) | ~22k | ~11.5k |
| | 4 × RHD 128 | 8 × Molex | 4 × SPI | ~16k | ~7k |

TABLE 3
List of components used for bi-directional recording/stimulation
system along with their costs in USD.^{a}

| Component | Cost | Component | Cost |
|---|---|---|---|
| RHS stimulation/recording | 11800 | Intan RHS 32ch | 1485 |
| controller | | Headstage | |
| RHS SPI cable | 265 | | |

^{a}Online prices as of August 2023

We claim:

1. An automated system for electrophysiologically interfacing with a biological material comprising:
a sterile platform comprising a plurality of recess features, each recess feature configured to support a biological material;
a microelectrode array (MEA) positioned in each of the plurality of recess features;
electrical interconnects configured to electrically interface with the biological material;
a downstream signal acquisition unit electronically connected to the biological material via the electrically conductive interconnects;
a plurality of lids, with each recess feature having one lid removably connected to removably seal the biological material from a surrounding environment;
a processor configured to receive instructions from a user for electrically interfacing with the biological material;
a robotic positioner comprising an end effector having:
a printed circuit board (PCB) interface to electrically connect to the MEA;
a lid grip to reversibly engage with each of the plurality of lids; and
an optical detector for aligning the PCB and the MEA;
a controller for implementing an electrophysiological interface scheme between the robotic positioner interface unit and at least one of the MEAs positioned in the recess feature, the electrophysiological interface scheme including:
transmitting a position signal to the robotic positioner to position the end effector and deploy the lid grip to engage the lid of a selected recess feature;
transmitting a removal signal to the end effector to remove the lid from the recess feature; and
transmitting an interface signal to the robotic positioner to align the PCB interface with the MEA and electrically connect the PCB interface with the MEA, thereby electrophysiologically interfacing with the biological material.

2. The automated system of claim 1, further comprising an optical light source connected to the end effector and configured to illuminate the MEA for the optical detector and alignment of the PCB interface with the underlying MEA.

3. The automated system of claim 1, wherein the biological material is positioned within a container and the container is positioned within the recess feature, wherein each container is removable and replaceable.

4. The automated system of claim 1, wherein the plurality of recess features is provided in an array having 12 or more of the recess features configured for independent electrophysiological interfacing with the biological materials in each of the recess features.

5. The automated system of claim 1, wherein the MEA comprises up to 512 electrode channels, with each electrode channel electronically connected to the PCB.

6. The automated system of claim 1, wherein the MEA comprises a spatial array of electrodes patterned on a biocompatible surface for directly supporting growth of a biological cell of the biological material.

7. The automated system of claim 1, wherein the MEA is a customized chip configured to match with the PCB that is a swappable PCB configured to an application of interest.

8. The automated system of claim 1, wherein the electrical interconnects are provided on the MEA and physically connect each microelectrode that is positioned in a central region, wherein the biological material is positioned to a corresponding plurality of contact pads positioned in an outer region perimeter orientation where the PCB interface makes electrical contact.

9. The automated system of claim 1, wherein the controller comprises a computing device, the computing device including: a processor, and memory communicatively coupled to the processor, and the computing device implements the electrophysiological interface scheme for the automated system.

10. The automated system of claim 9, wherein the memory includes a non-transitory computer readable medium storing processor-executable instructions encoded as software, which, when executed by the processor, cause the processor to implement the control scheme for the system.

11. The automated system of claim 1, wherein the electrophysiological interface scheme:
records electrical output from the biological material via the MEA;
provides an electrical activation input to the biological material via the MEA; and/or
records electrical output from the biological material via the MEA and provides an electrical activation input to the biological material via the MEA.

12. The automated system of claim 1, wherein the electrophysiological interface scheme further includes:
removing the biological material from the recess feature; and
inserting a different biological material into the recess feature;
so that the automated system comprises a fully reconfigurable platform.

13. The automated system of claim 1, wherein the electrophysiological interface scheme further includes:
controlling one or more cell culture parameters to support or maintain growth of the biological materials in the recess features.

14. The automated system of claim 1, wherein the biological material is selected from the group consisting of: neuronal cells, cardiac cells, stem cells, brain cells, tissue slices, skeletal muscle cells, retinal ganglion cells, multi-cell type co-cultures including neuromuscular junctions and the combination of multiple neuronal subtypes, a bioengineered tissue, and ex vivo slices of a biological tissue.

15. The automated system of claim 1, further comprising an optical interface, a fluidic interface, or both an optical and a fluidic interface.

16. The automated system of claim 15, further comprising an optical source operably connected to the controller for implementing the optical interface, including for detecting an optical marker and/or activation of an optical probe.

17. The automated system of claim 15, wherein the fluidic interface comprises an inlet tube fluidically connected to the plurality of recess features for introducing cell media from a source of cell media to the biological material in the recess feature.

18. The automated system of claim 1, wherein the recess features is cylindrical having a depth and a diameter, wherein the depth is between 0.5 cm and 3 cm and the diameter is between 1 cm and 15 cm.

19. A method of electrophysiologically interfacing with a biological tissue, the method comprising the steps of:
inserting a biological material onto a microelectrode array (MEA) positioned within a sterile platform recess feature;
covering the inserted biological materials in the sterile platform recess feature with a lid;
positioning the sterile platform into an incubation chamber;
controlling the incubation chamber to maintain viability of the biological material for a culture time period and to thereby electrically connect the biological material with at least one microelectrode of the microelectrode array;
providing an electrophysiological interface scheme to control a robotic positioner having an end effector with a lid grip and a PCB interface for
removing at least one lid with the end effector lid grip from at least one underlying recess feature to generate a lid in a lid removed configuration and thereby provide an opening to access the biological material;
aligning the PCB interface with the MEA;
interfacing the robotic positioner PCB interface with the biological material by contacting the PCB interface with the MEA for an interface time period;
terminating the interfacing by removing the PCB interface unit from the MEA;
directing the robotic positioner lid grip to contact the lid in the lid removed configuration and move the lid over the underlying recess feature;
depositing the lid to cover the underlying recess feature and thereby seal the biological material from the surrounding environment and generate the lid in a lid sealed configuration.

20. The method of claim 19, wherein the steps are repeated for one or more additional recess features.

21. The method of claim 19, further comprising the step of:
controlling a cell culture parameter to maintain viability of the biological material, wherein the cell culture parameter is selected from the group consisting of: a media flow rate; a temperature; a humidity; a CO₂ concentration; and any combination thereof.

22. The method of claim 19, further comprising the step of directing via the electrophysiological interface scheme the robotic positioner to replace at least one biological material within a recess feature with a replacement biological material.

23. A system for electrophysiologically interfacing with a biological material comprising:
a sterile platform;
a microelectrode array (MEA) supported by the sterile platform, the MEA comprising:
a plurality of electrodes positioned in a central region of the MEA configured to support and electrically connect with the biological material;
a plurality of contact pads positioned around a perimeter region of the MEA;
a plurality of electrical interconnects, wherein each of the plurality of electrodes is connected to a unique contact pad by one of the electrical interconnects;
a PCB interface, wherein the PCB interface is configured to reversibly connect to the plurality of contact pads and configured to provide electrical connection between the PCB interface and the biological material; and
a downstream signal acquisition unit electronically connected to the PCB interface.

24. The system of claim 23 further comprising:
a cover to cover the MEA supporting the biological material;
a downstream signal acquisition unit;
a cable that electronically connects the PCB interface to the downstream signal acquisition unit;
wherein:
the PCB interface is connected to the end effector to provide a PCB interfaced with the biological material and a PCB not-interfaced with the biological material condition;
the cover is configured for movement via the end effector.

25. The system of claim 23, wherein the PCB interface is a swappable PCB and the MEA is a custom chip having a user-selected number of contact pads, with the swappable PCB selected based on the number of contact pads, including a number of contact pads ranging from between 12 and 600.
